SEQUENCE LISTING (8 sequences)

SEQ ID NO: 1 - protein, 76 aa, Homo sapiens [sequence omitted]

SEQ ID NO: 2 - protein, 76 aa, Artificial Sequence
  Artificially synthesized ubiquitin with N-terminal Met replaced with Leu in order to avoid oxidation of the Met
LQIFVKTLTGKTITLEVEPSDTIENVKAKIQDKEGIPPDQQRLIFAGKQLEDGRTLSDYNIQKESTLHLVLRLRGG

SEQ ID NO: 3 - protein, 31 aa, Artificial Sequence
  Artificially-synthesized peptide sequence for use together with (Ub1-45)-SR in the 2-fragment synthesis of ubiquitin
AGKQLEDGRTLSDYNIQKESTLHLVLRLRGG

SEQ ID NO: 4 - protein, 45 aa, Artificial Sequence
  Synthetic peptide sequence for use together with Ub46-76 in the 2-fragment preparation of ubiquitin
LQIFVKTLTGKTITLEVEPSDTIENVKAKIQDKEGIPPDQQRLIF

SEQ ID NO: 5 - protein, 49 aa, Artificial Sequence
  Synthetic peptide sequence for use together with (Ub1-27)-SR in the 2-fragment preparation of ubiquitin
AKIQDKEGIPPDQQRLIFAGKQLEDGRTLSDYNIQKESTLHLVLRLRGG

SEQ ID NO: 6 - protein, 27 aa, Artificial Sequence
  Synthetic peptide sequence for use together with Ub28-76 in the 2-fragment preparation of ubiqutine
LQIFVKTLTGKTITLEVEPSDTIENVK

SEQ ID NO: 7 - protein, 31 aa, Artificial Sequence; identical to SEQ ID NO: 3
  Synthetic peptide sequence for use together with Ub46-76 and (Ub1-27)-SR in the 3-fragment preparation of ubiquitin

SEQ ID NO: 8 - protein, 125 aa, Artificial Sequence
  125 amino acid synthetic ubiquinated peptide
PEPAKSAPAPKKGSKKAVTKAQKKDGKKRKRSRKESYSVYVYKVLKQVHPDTGISSKAMGIMNSFVNDIFERIAGEASRLAHYNKRSTITSREIQTAVRLLLPGELAKHAVSEGTKAVTKYTSSK

(19) United States
(12) Patent Application Publication
(10) Pub. No.: US 2013/0143251 A1
(43) Pub. Date: Jun. 6, 2013

(54) EXPEDITIOUS SYNTHESIS OF UBIQUITINATED PEPTIDE CONJUGATES

(71) Applicants: Ashraf Brik, Be'er-Sheva (IL); Ajish Kumar, Kochi (IN); Leslie Erlich, Hertzliya (IL); Liat Spasser, Hertzliya (IL)

(72) Inventors: Ashraf Brik, Be'er-Sheva (IL); Ajish Kumar, Kochi (IN); Leslie Erlich, Hertzliya (IL); Liat Spasser, Hertzliya (IL)

(73) Assignee: BEN-GURION UNIVERSITY OF THE NEGEV RESEARCH AND DEVELOPMENT AUTHORITY, Beer-Sheva (IL)

(21) Appl. No.: 13/701,731

(22) PCT Filed: Jun. 1, 2011
(86) PCT No.: PCT/IL11/00427

§ 371 Date: Feb. 15, 2013

Related U.S. Application Data
(60) Provisional application No. 61/344,136, filed on Jun. 1, 2010.

(51) Int. Cl. C07K 1/04

(52) U.S. Cl. CPC C07K 1/04
USPC 435/24; 530/334; 530/300

(57) ABSTRACT

The present invention discloses a process for preparing ubiquinated peptide conjugates comprising a ubiquitin peptide residue UR attached at its C-terminus to a substrate peptide via a native isopeptide bond, this process comprising combining Native Chemical Ligation (NCL) and solid phase peptide synthesis (SPPS). Further are disclosed ubiquinated peptide conjugates containing a native isopeptide bond, as well as various uses thereof.

A novel strategy for the synthesis of ubiquitinated peptide conjugates is presented, based on solid phase peptide synthesis (SPPS) coupled with native chemical ligation (NCL).

Chemical synthesis of ubiquitinated peptide conjugates and proteins offers an excellent opportunity for preparing these conjugates due to the precise control over the ubiquitination site and the large quantities of the desired product which could be obtained. Moreover, chemical methods do not rely on the E2 and E3 enzymes that are very specific to each protein target. These enzymes, in addition to the E1 enzyme, which activates ubiquitin (Ub) C-terminal acid, constitute the enzymatic machinery that is used to assemble the isopeptide bond between the ε-amine of a lysine residue in the target protein and the C-terminal of Ub (C. M. Pickart, Annu. Rev. Biochem., 2001, 70, 503). In a similar manner other ubiquitin like modifiers (Ub1) are also tagged to the lysine side chain leading to a variety of biochemical processes including, but not limited to, DNA-damage response, cell-cycle process, and regulation of cell survival (O. Krescher et al., Ann. Rev. Cell, Develop. Biol. 2006, 22, 159). As a result, protein modification with Ub and Ub1 are very complex posttranslational events wherein chemical approaches could offer a complementary way to the biochemical methods aiming at understanding the role of these modifications on protein function. Thus, there is an urgent need for efficient and readily available methods to generate sufficient amounts of highly homogeneous ubiquitinated peptides and protein reagents. These conjugates could serve as assays, antigens for developing linkage-specific antibodies, and as substrates for enzymatic elaboration to prepare a ubiquitin chain linked to peptide or protein targets.

Recently, several groups have introduced chemical methods for linking Ub and Ub1 to peptides or proteins via non-native isopeptide bonds to generate enzymatically stable ubiquitinated conjugates (See for example Weikart, N. D., and Mootz, H. D. (2010), ChemBioChem 11, 774-777).

While these methods facilitated the synthesis of ubiquitinated peptides and proteins for a variety of important studies, the absence of a native and reversible isopeptide bond precludes the use of these conjugates to investigate the mechanisms by which the dynamics of ubiquitination (i.e ubiquitination-deubiquitination) regulate protein function and biological processes.

Site-specific ubiquitination of peptides and proteins via formation of a native isopeptide bond is now possible thanks to the work of several groups, including that of the present inventors, as shown in Scheme 1. Throughout the scheme, AA is for amino acid, K* stands for the modified Lys, R=—CH₃ and R′=—(CH₂)₃CONH—CH₃.

The first route was introduced by Muir and coworkers, who relied on the use of a photocleavable auxiliary (Scheme 1(C), see Chatterjee, C. et al. (2007), Angew. Chem. Int. Ed. 46, 2814-2818). The present inventors as well as Ovaa's group have previously introduced the use of δ-mercaptolysine to promote isopeptide formation (Scheme 1(A), see a) Kumar, K. S. A. et al. (2009), Angew. Chem. Int. Ed. 48, 8090-8094 and b) WO 2010/131962 to Ovaa et al.)). Similarly, the Liu group devised the γ-mercaptolysine analog to assist isopeptide formation in addition to backbone ligation (Scheme 1(B), see Yang, R. et al. (2009), J. Am. Chem. Soc. 131, 13592-13593). More recently, Chin and Komander developed genetically encoded orthogonal protection and activated ligation in the presence of AgNO₃/HOSu in DMSO to generate the isopeptide linkage in a site-specific manner (Scheme 1(D), see Virdee, S. et al. (2010), Nat. Chem. Biol. 6, 750-757).

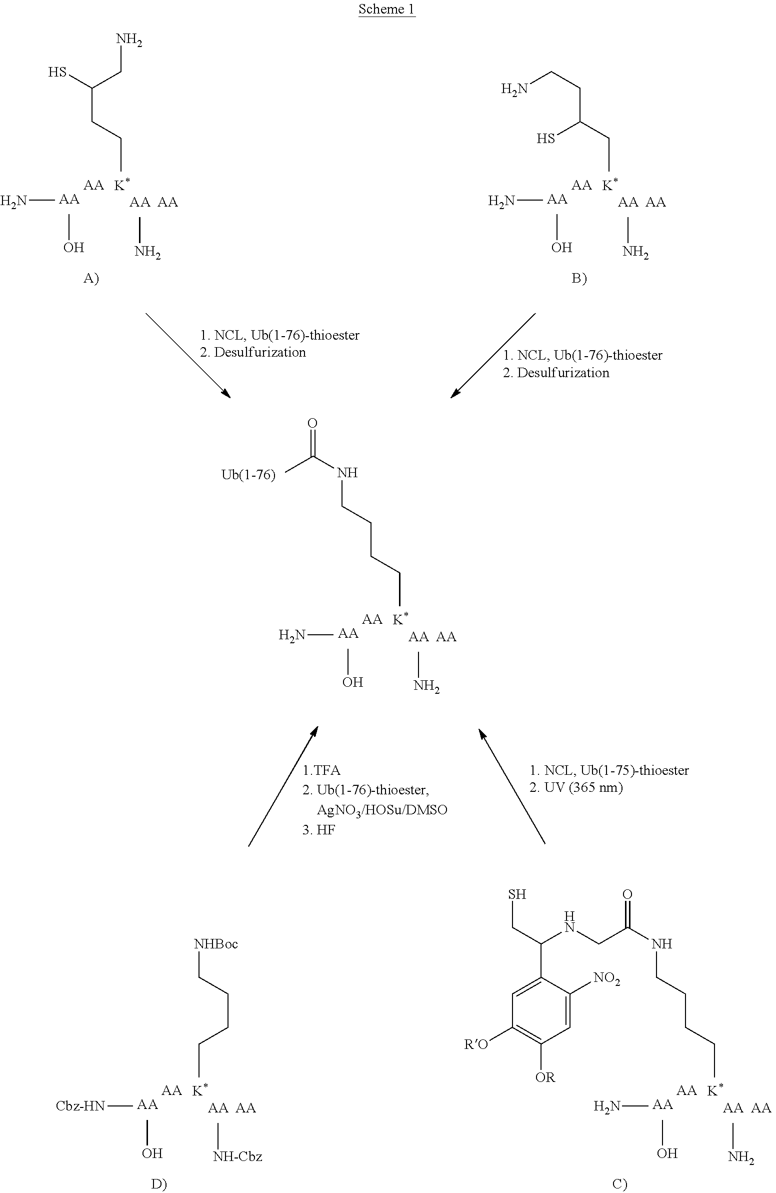

In these studies, multi-step syntheses (8-19 steps) are needed to prepare the desired auxiliary or the thiolysine analogs. In many cases, such as the synthesis of ubiquitinated peptides, these processes can be tedious and very limited to the synthetic community, rendering these methods inaccessible to many applications.

Therefore, there is a need for novel methods to prepare Ub peptides and proteins, that would overcome these disadvantages and would further be suitable for obtaining large quantities of the desired products.

The ubiquitination process is of special interest since the attachment of a ubiquitin (Ub or monoUb) or polyubiquitin (polyUb) chain to a protein target is involved in a wide range of cellular processes in eukaryotes, leading to a variety of molecular signals, e.g., regulation of protein degradation and DNA repair, with the outcome depending on the nature of the ubiquitination (namely polyubiquitination vs. monoubiquitination), on the specific site of attachment on the protein and on the Ub molecule. Ubiquitination is a reversible posttranslational modification, in which the removal of the Ub molecule is achieved by a family of enzymes known as deubiquitinases (DUBs).

DUBs can remove Ub or polyUb from proteins, process Ub precursors, and disassemble unanchored polyUb chains. Approximately 100 DUBs encoded in the human genome are involved in a variety of regulatory processes such as cell-cycle progression, tissue development and differentiation, and therefore they may represent new therapeutic targets. Indeed, several DUBs have been implicated in different diseases including neurological disorders, infectious diseases and cancer. For example, a genome-wide RNA interference identified the Ub specific proteases 7/herpesvirus-associated ubiquitin specific-peptidase (USP7) as a promising target in cancer. UCH-L3 is another DUB that is known to play an important role in programmed cell death, a process which is implicated in a number of human diseases. For example, over expression and increase of UCH-L3 activity is reported in multiple types of cancer cells suggesting that its activity might be required for cancer cell survival. Until now, there has been no general knowledge of any inhibitors that are both suitable against DUBs and that have made it to the clinical trial stage. This is mainly due to the absence of highly efficient, sensitive yet general high throughput screening (HTS) assay to identify potential inhibitors against a DUB of interest. One of the reasons for the lack of such assays is the synthetic difficulties to prepare systems that take into consideration the relatively complex structure of the natural Ub-peptide substrates. So far, several strategies have been introduced trying to overcome these limitations by avoiding the construction of the native isopeptide bond (T. Fekner, X. Li, M. K. Chan, ChemBioChem, 2011, 12, 21-33). However, lacking the native isopeptide bond, the obtained assays have low sensitivity and therefore have limited applicability for most DUBs. Thus, there is a need to develop novel high throughput assays that will be suitable for the screening and search of suitable inhibitors against important deubiquitinases (DUBs).

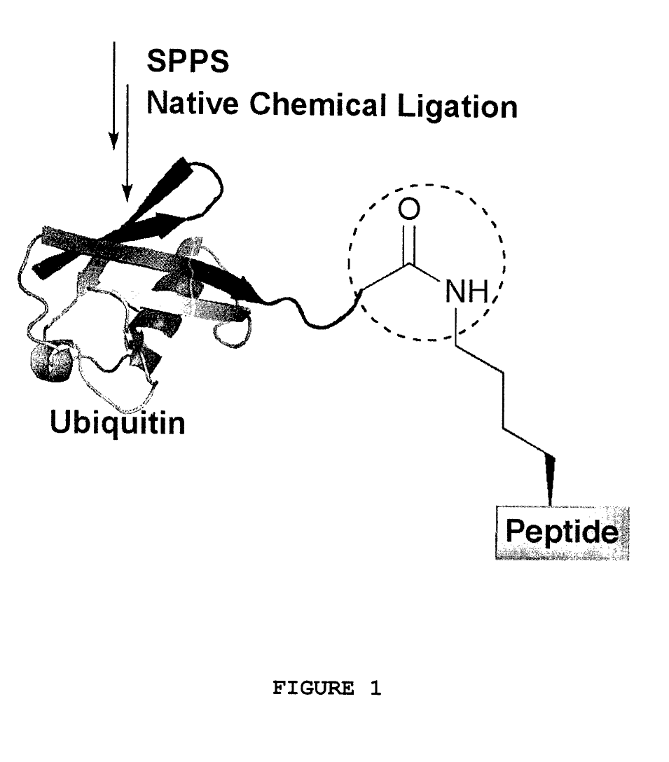

The present inventors have now developed a novel method for the expeditious synthesis of ubiquitinated peptides having native isopeptide bonds, this method relying on the modification of solid phase peptide synthesis (SPPS) combined with native chemical ligation (NCL), as shown in FIG. 1.

Using these methods, the inventors have successfully and expeditiously prepared several ubiquitinated peptide conjugates. Among the Ub-peptide conjugates that have been prepared are those derived from p53, di-Ub and H2B. The latter ubiquitinated peptide is a key precursor in the synthesis of mono-ubiquitinated H2B.

The inventors have also successfully demonstrated that the Ub-peptide conjugates obtained by this method have natural 3-D structure, since the obtained Ub-peptides proved to be successful substrates for natural deubiquitinases (DUBs). For example, the inventors have shown the successful enzyme activity of ubiquitin C-terminal hydrolase (UCH-L3) to hydrolyze the isopeptide bond for the Ub-peptides 22-24, resulting in separate Ub and peptide products (see Example 4).

Yet further, the inventors have developed and tested the application of this new method for conducting novel high throughput assays that are suitable to detect inhibitors against deubiquitinases (DUBs) (see Example 5) by incorporating fluorescent labels both on the ubiquitin peptide and in the substrate peptide, and have shown the existence and disappearance of fluorescence after treating this doubly-labeled ubiquinated peptide conjugate with a DUB.

The method of the present invention for the rapid synthesis of ubiquitinated peptides is depicted in exemplary Scheme 2 below. In this approach, SPPS is used to build the target peptide linked through an isopeptide bond to a C-terminal fragment of Ub (for example Ub₄₆₋₇₆), such as fragment 1 of scheme 2.

According to preferred embodiments of the present invention, this peptide also bears N-terminal Cys amino acid, instead of the original Ala₄₆ residue, which is introduced to facilitate the NCL step with a complimentary Ub thioester fragment (such as Ub(₁₋₄₅) thioester 2 of scheme 2).

As used herein, the term “thioester”, interchangeably used with the term “thioloester”, refers to a moiety represented by —COSR, often connected to a peptide.

The term “thioester peptide”, “peptide thioester” or “polypeptide in its thioester form” may be represented as “peptide-α-COSR”. The R group in this case may be any number of groups, including 1-15 C functionalized alkyl, straight or branched, 1-15 C aromatic structures, 1-4 amino acids or derivatives thereof, preferably wherein the R group is selected such that the peptide-α-COSR is an activated thioester.

The preparation of the peptide fragments is preferably conducted by SPPS, according to techniques known to those skilled in the art.

Preferably, the SPPS is an Fmoc synthesis, but Boc synthesis can also be used.

The term “complimentary fragment” as used herein refers to a peptide fragment that, when attached to another peptide fragment forms the complete sequence of the desired polypeptide. The complimentary fragment can be made in one or more steps, as required.

Thus, the process follows with a ligation step between ubiquitin fragment peptides 1 and ubiquitin thioester 2, which results in the full-length ubiquinated peptide conjugate 3.

Then, optionally, the Cys₄₆ amino-acid is converted to the native Ala₄₆, applying well-established desulfurization conditions (Yan, L. Z., and Dawson, P. E. (2001), J. Am. Chem. Soc. 123, 526-533) to furnish the unmodified ubiquitinated peptide 4 of scheme 2. However, it should be noted that this step is only necessary to obtain the natural ubiquitin structure, and that for many other applications, a modified ubiquitin may be suitable and useful.

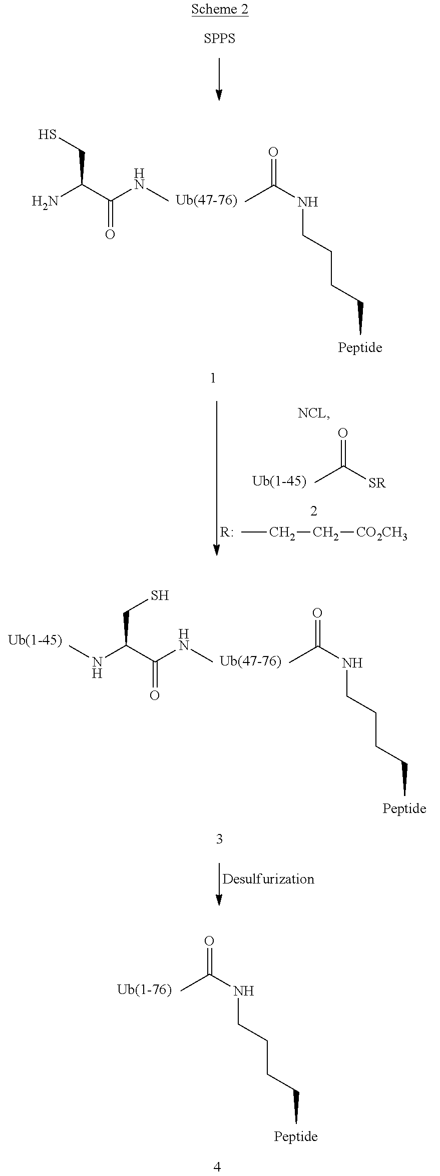

To implement the above-described strategy, the inventors have designed a synthetic approach to obtain a branched peptide pre-1, which is a challenging yet essential precursor to the Ub-fragment peptide 1 in the above-described synthetic strategy.

This species is prepared during the SPPS of its target peptide, by designing and incorporating within the sequence a modified Lysine amino acid (K*) that is protected by two different protecting groups: P, a “regular” protecting group for either Boc SPPS or Fmoc SPPS, and OP, an orthogonal protecting group, as shown in Scheme 3 below:

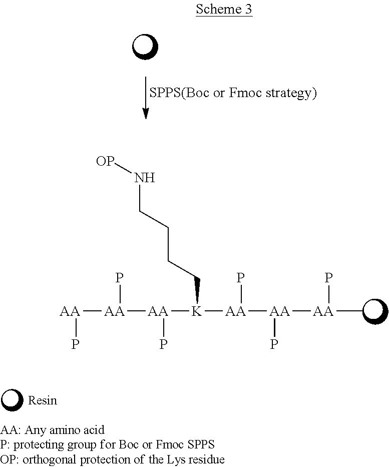

According to Scheme 3, the peptide is grown by SPPS, either by FMOC or Boc chemistry, whereas the modified Lys (K*) amino acid is introduced as a species protected by two different and carefully-chosen protecting groups, P and OP groups, thereby creating a branching point onto which, at a later stage, the Ubiquitin peptide can be grown on, or attached to.

After creating this branching point, either the substrate peptide is grown by deprotection of the P protecting group of the terminal-N and conducting SPPS, or a fragment of the uniquitin peptide can be grown by selectively removing the OP group and conducting SPPS.

Thus, according to a first aspect of the invention, there is provided a process for preparing ubiquinated peptide conjugates comprising a ubiquitin peptide residue UR attached at its C-terminus to a substrate peptide via a native isopeptide bond, said process comprising combining Native Chemical Ligation (NCL) and solid phase peptide synthesis (SPPS), as follows:
i) Conducting Solid Phase Protein Synthesis (SPPS) to obtain peptide fragment A linked to a solid support:

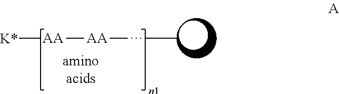

wherein this peptide fragment A contains n₁ amino acids (AA), n₁ being an integer≧0, this peptide fragment A N-terminating with a modified Lys amino acid K*:

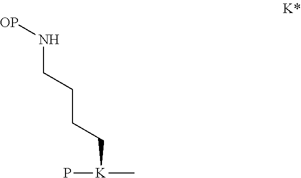

Whereas:
   P is a terminal-amine protecting group, and
   OP is an orthogonal E-amine protecting group,
Further wherein K is a Lys amino acid backbone of the formula —(NHCH—C═O)—; this K amino acid forming a branching point for further elongation of the peptide fragment A;
ii) Selectively removing the ε-amine orthogonal protecting group OP, and adding by SPPS up to m₁ amino acids belonging to a Ubiquitin peptide at the ε-amine of the Lys amino acid in peptide fragment A, optionally further adding by SPPS up to n₂ amino acids to the terminal amine of the Lys amino acid in peptide fragment A, wherein n₂ is an integer≧0, m₁ is an integer≧1 and n₁+n₂+m₁+1 is an integer≧80,
further whereas the adding of up to n₂ amino acids to the terminal amine is conducted either before removing the OP group, or after the adding of up to m₁ amino acids belonging to a Ubiquitin peptide at the ε-amine, thereby obtaining a ubiquinated-fragment peptide conjugate B linked to the solid support:

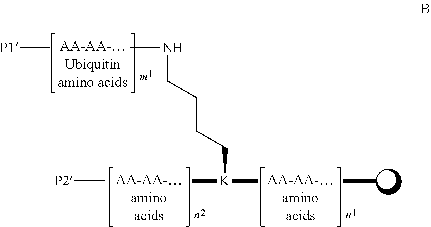

wherein the ubiquinated-fragment peptide conjugate B contains up to m₁ amino acids belonging to a Ubiquitin peptide at the ε-amine of the Lys amino acid, up to n₁ amino acids attached to the solid support at the C-terminus of the Lys amino acid, prior to the branching point, and up to n₂ amino acids attached at the N-terminus of the Lys amino acid, after the branching point, further wherein the ubiquinated-fragment peptide conjugate B contains n₁+n₂+m₁+1 amino acids, and N-terminates with P1′ and P2′ amine-protecting groups;
iii) Cleaving the ubiquinated-fragment peptide conjugate B from the solid support, to obtain a free ubiquinated-fragment peptide C:

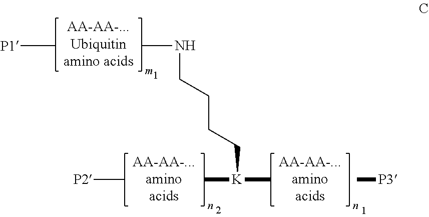

, wherein the ubiquinated fragment peptide conjugate C contains n₁+n₂+m₁+1 amino acids, and terminates with P1′ and P2′ amine-protecting groups and a P3′ carboxy-protecting group;
According to preferred embodiments of the present invention, P3′ is selected from hydrogen, N-acylurea (Nbz) and a latent thioester Functionality (LTF) residue having the general structure of Formula V:

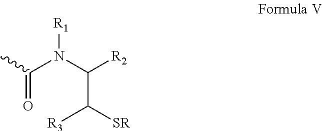

Wherein:
R is either hydrogen or a thiol protecting group;
R₁ is selected from the group consisting of: hydrogen, C1-C3 alkyl, C1-C3 alkyl-COON, C1-C3 alkyl-CONH₂, C1-C3 alkylene-CONH₂, C1-C3 alkylene-CO₂H, SO₂-alkyl; SO₂-alkyl-CONH₂, benzyl and derivatives thereof, alkyl-nitrile and alkyl-halogens;
R₂ and R₃ are selected from the group consisting of: hydrogen, CO₂H, CH₂CO₂H, —CH₂OH, CONH₂, CH₂—CONH₂ and CH₂NH₂. and N-protected derivatives thereof.
Depending on the required Ubiquitin sequence (complete sequence needed or not) and on the target peptide to be attached to the ubiquitin peptide, additional fragments are to be added by Native Chemical Ligation (NCL).
Thus, following the cleavage from the solid support, follows:
iv) Optionally further elongating the ubiquinated fragment peptide conjugate C by ligating it with one or more additional peptides, by Native Chemical Ligation (NCL), to obtain a ubiquinated peptide conjugate D:

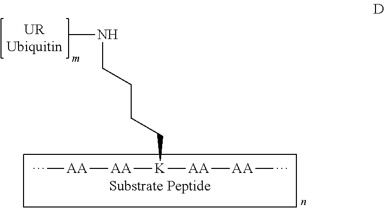

wherein the ubiquinated peptide conjugate D contains m amino acids of the ubiquitin peptide attached via an isopeptide bond to a substrate peptide containing n amino acids, such that m≧m₁ and n≧n₁+n₂+1.
Any person skilled in the art would understand that step ii, namely obtaining ubiquinated-fragment peptide conjugate having general structure B from peptide fragment having general structure A, can be conducted in any number of ways.
For example, according to one preferred embodiment of the invention, first the OP group is selectively removed, to be followed by SPPS of the ubiquitin fragment containing ml amino acids to obtain fragment A1:

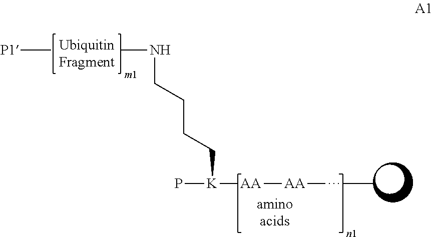

and only then is SPPS conducted on the terminal amine of the peptide backbone, to obtain ubiquinated-fragment peptide conjugate having general structure B.
According to another preferred embodiment of the invention, first-SPPS conducted on the terminal amine of the peptide having general structure Backbone to obtain fragment A2:

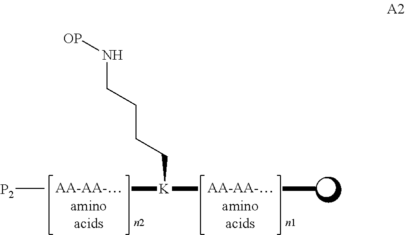

and only then is the OP group selectively removed, to be followed by SPPS of the ubiquitin fragment containing m1 amino acids, to obtain ubiquitinated-fragment peptide having general structure B.

The term “peptide” or “polypeptide” as used herein refers to a sequential chain of amino acids linked together via peptide bonds and encompasses an amino acid chain of any length. If a single polypeptide can function as a unit, the terms “polypeptide” and “protein” may be used interchangeably, however, in general, the term includes peptides, proteins, fusion proteins, oligopeptides, cyclic peptides, and polypeptide derivatives.

The term “substrate peptide” is used interchangeably with the term “target peptide” and refers to a peptide that has a specific binding with a species, such as an enzyme, a receptor, an agonist, an antibody, an antigen, a lectin or a carbohydrate. The term “specific binding” is defined further below.

According to one preferred embodiment of the present invention, the substrate peptide can be another ubiquitin peptide or a fragment thereof, thereby obtaining a ubiquinate peptide which is in fact a di-ubiquitine polypeptide.

Thus, according to an additional aspect of the invention, there is now provided the use of the ubiquinated peptide conjugates described hereinabove in the preparation of substrates for enzymatic elongation of ubiquitin.

In another preferred embodiment of the present invention, the substrate peptide according to the present invention has a specific binding affinity with one or more natural deubiquitinases (DUBS).

The suitability of the ubiquinated peptide conjugate to form a substrate for a particular Dub can be tested by comparing the enzymatic activity of this DUB with or without the synthesized conjugate, as was shown by the inventors in an exemplary demonstration in the case of the enzyme UCH-L3 (Example 4).

The term “peptide fragment” is used interchangeably with the terms “polypeptide fragment” or “polypeptide segment” and refers to a peptide or polypeptide, having either a completely native amide backbone or an unnatural backbone or a mixture thereof, ranging in size from 2 to 1000 amino acid residues, preferably from 2-99 amino acid residues, more preferably from 10-60 amino acid residues, and most preferably from 20-40 amino acid residues. Each peptide fragment can comprise native amide bonds or any of the known unnatural peptide backbones or a mixture thereof. Each peptide fragment can be prepared by any known synthetic methods, including solution synthesis, stepwise solid phase synthesis, segment condensation, and convergent condensation.

The term “N-terminal” is interchangeably used with “N-terminus” or “N-terminus amino acid” and refers to mean, as used herein, the amino acid whose carboxyl group participates in the formation of the peptide bond, but which has a free amino group. In a linear peptide, the N terminus is conventionally written to the left.

The term “free amino group” as used herein refers to an amino acid that is partially protected or is not protected at all, and is able to participates in the formation of the peptide bond.

The term “C-terminal” is interchangeably used with “C-terminus” or “C-terminus amino acid” and refers to mean, as used herein, the amino acid whose amino group participates in the formation of the peptide bond, but which still has a free carboxyl group. In a linear peptide, the C-terminus is conventionally written to the right.

The term “solid phase peptide synthesis” (SPPS), pioneered by Robert Bruce Merrifield refers to the commonly-accepted method for creating peptides and proteins in the lab in a synthetic manner. SPPS is based on repeated cycles of coupling-wash-deprotection-wash. The free N-terminal amine of a peptide attached to the solid-phase is coupled with a single N-protected amino acid unit. This unit is then deprotected, revealing a new N-terminal amine to which a further amino acid may be attached. SPPS proceeds in a C-terminal to N-terminal direction.

SPPS is limited by yields, and typically peptides and proteins in the range of 80 amino acids are pushing the limits of synthetic accessibility. Longer lengths can be accessed by using native chemical ligation to couple two peptides together with quantitative yields.

Therefore, according to the process of the present invention, it is necessary to design the process such that the number of amino acids introduced into the ubiquinated peptide conjugate by SPPS, namely n₁+n₂+n₃+1, shall remain just under 80 amino acids. Preferably, their number shall be smaller than 70 amino acids, more preferably in the range of 50-70 amino acids. Thus, for example, if the substrate peptide contains 5 amino acids before the branching Lys amino acid, the ubiquitin segment that is prepared by SPPS can be longer than that prepared if the initial substrate length before the peptide is 30 amino acids long. Another factor determining the length of each segment being elongated by SPPS, is the necessity to cut the long ubiquitin chain in positions that would be appropriate for later native chemical ligation.

The term “solid substrate” is used interchangeably with the terms “solid Phase” or “solid support” and refers to a material having a surface and which is substantially insoluble when exposed to organic or aqueous solutions used for coupling, deprotecting, and cleavage reactions.

Examples of solid support materials include glass, polymers and resins, including polyacrylamide, PEG, polystyrene PEG-A, PEG-polystyrene, macroporous, POROS™, cellulose, reconstituted cellulose (e.g. Perloza), nitrocellulose, nylon membranes, controlled-pore glass beads, acrylamide gels, polystyrene, activated dextran, agarose, polyethylene, functionalized plastics, glass, silicon, aluminum, steel, iron, copper, nickel and gold. Such materials may be in the form of a plate, sheet, petri dish, beads, pellets, disks, or other convenient forms.

Some of the examples and embodiments described herein refer to resins, which are a type of solid support, and one of ordinary skill in the art would understand that such examples are not meant to be limited to resins, but to solid phases in general.

As used herein, the term “ubiquitin” is used interchangeably with the terms “ubiquitin peptide” or “ubiquitin polypeptide” or Ub, and includes within its scope all known as well as unidentified eukaryotic Ub homologs of vertebrate or invertebrate origin. Examples of Ub polypeptides as referred to herein include the human Ub polypeptide that is encoded by the human Ub encoding nucleic acid sequence (GenBank Accession Numbers: U49869, X04803) as well as all equivalents.

For example, natural human Ub protein has the following sequence, containing the following 76 amino acids: MQIFVKTLTGKTITLEVEPSDTIENVKAKIQDKEGIPPDQQRLIFAGKQLEDGRTLSDYNIQK BESTLHLVLRLRGG.

Therefore, according to one preferred embodiment of the present invention, the ubiquitin polypeptide is a natural ubiquitin polypeptide.

However, as used herein, the term “ubiquitin” (Ub) also includes modified ubiquitin polypeptides.

The term “modified Ub” as used herein refers to a ubiquitin peptide, wherein one or more of the 76 native amino acids comprising it, is replaced or substituted by another amino acid. This amino acid can be either natural or unnatural.

For example, in the ensuing examples, an equivalent sequence to natural Ub was synthetically prepared, replacing the Met amino acid with a Leucine amino acid (namely to obtain the following sequence: LQIFVKTLTGKTITLEVEPSDTIENVKAKIQDKEGIPPDQQRLIFAGKQLEDGRTLSDYNIQK ESTLHLVLRLRGG), thereby avoiding oxidation of the Met. Similarly, another equivalent is obtained by replacing the Met with nurleucine (Nle). However, the invention also works with the original Met amino acid.

Additional examples to modified Ub include replacing at least one other natural amino acids by at least one non-natural or modified amino acid, for example replacing the Leu amino acids in positions 28 or 46 by a 1,2 thioamine containing amino acid, such as mercaptolysine derivatives, or by introducing a labeled amino acid, or an amino acid linked to a specific reagent etc. Additional useful modifications can be envisioned by a person skilled in the art and are therefore included in the scope of this invention.

Therefore, according to another preferred embodiment of the present invention, the ubiquitin peptide is a non-natural, or modified, ubiquitin peptide.

According to yet another preferred embodiment of the present invention, the ubiquitin polypeptide is a modified ubiquitin peptide.

The ubiquitin according to the present invention includes both mono-ubiquitin and poly-ubiquitin. In other words, the ubiquitin may appear as having either one or several ubiquitin monomers.

The term “Ub monomer”, used interchangeably with the term “Ub unit”, as used herein, refers to a 76-amino acid sequence of ubiquitin, either natural or modified.

Furthermore, this term includes ubiquitin-like-modifiers (ULM), also termed “ubiquitin-like” or “Ub1” protein modifiers. This term, as used herein, refers to the group of small proteins that are subject to conjugation machinery similar to that for ubiquitination. Examples of Ub1 protein modifiers include NEDD8, ISG15, SUMO1, SUMO2, SUMO3, APG12, APG8, URM1, Atg8, URM1, HUB1, FUB1, FAT10, UBL5, UFM1, MLP3A-LC3, ATG12, as well as other Ub1 protein modifiers yet to be identified.

The term “ubiquitin peptide residue” UR, refers to part or all of the ubiquitin amino acid sequence that is attached to the substrate peptide via an isopeptide bond.

The term “ubiquitin fragment” refers to a residue of the full or natural ubiquitin fragment that contains less than the 76 amino acids comprising the natural ubiquitin.

It should be noted that the term “ubiquitin peptide” as used herein, also includes conjugates of ubiquitin peptides with additional peptides or fragments thereof.

The term “ubiquinated peptide” as used herein is used interchangeably with the term “ubiquinated peptide conjugate” and refers to a chemical conjugate, namely a covalent linking, between a ubiquitin peptide and a substrate peptide via a native isopeptide bond.

The term “native isopeptide bond” is used interchangeably with the term “iso peptide bond” and is used herein to refer to a natural linkage between the ubiquitin peptide and a substrate peptide, as well as between two or more ubiquitin monomers. In particular, the ubiquitin attaches via an iso-amide bond formed between the C-terminal glycine residue of ubiquitin and a lysine side chain of the substrate protein, hence forming an isopeptide bond.

The term “Native Chemical Ligation” (NCL) as used herein refers to chemoselective reactions involving ligation of a first unprotected amino acid, peptide or polypeptide and a second unprotected amino acid, peptide or polypeptide resulting in the formation of an amide bond having a backbone structure indistinguishable from that of a peptide or polypeptide occurring in nature or via recombinant expression. The Native Chemical Ligation is conducted according to techniques known to those skilled in the art.

Although several ligation reactions can be conducted to obtain the final peptide from fragments comprising it, either on the main backbone of the polypeptide, or via side chains thereof, preferably, the ligation reaction is between a Cysteine amino acid on the C-terminal of the polypeptide and a thioester or thioester equivalent, such as Nbz, linked to the N-terminal of the substrate peptide.

Therefore, the fragments are preferably prepared such that the N-terminal of the polypeptide would be in a thioester form or as a thioester equivalent, and that the C-terminal of the polypeptide would contain a Cys terminal amino acid, or an equivalent thereof.

Thus, according to preferred embodiments of the present invention, the additional peptides being added during the native chemical ligation in step iv, are thioester peptides or thioester-equivalent peptides.

The term “thioester equivalent” refers to a molecule that is a precursor to a thioester; namely, that it can be chemically turned into a thioester, for example upon reaction with an external thiol. Both the thioester and its analog or equivalent need to be stable at pH 6-8, and further need to be able to react with a Cys amino acid, or an equivalent thereof. An exemplary thioester equivalent is N-acylurea (Nbz), but other compounds may be suitable.

The Cys amino acid, which is used to affect the NCL, can be turned into Ala amino acid by desulfurization, either after the ligation step, in order to revert to the native polypeptide structure.

It should be noted that while the process for preparing Ubiquitin peptides, as described hereinbelow, can be conducted by “elongating” one long chain of the Ub monomer/unit on the solid support to which the substrate peptide is attached, this process is less desirable for the 76-amino acid-long ubiquitin, having lower yields and is generally less convenient, since any modification in this long chain requires a complete synthesis of the entire 76-amino acid chain. Therefore, although complete synthesis of Ub peptides has been conducted by the inventors, using native chemical ligation (NCL) of shorter fragments of the Ub monomer is a preferred embodiment of the present invention.

Since one preferable way of conducting NCL is based on a reaction of a thioester fragment with a Cys amino acid residue on the second peptide fragment, and since the natural Ub sequence has no Cys amino acids, NCL of ubiquitin fragments is preferably conducted in the positions containing Ala amino acids (namely positions 28 and 46), by chemically introducing one or more Cys amino acids into one or more of those positions, whereas at some stage after the ligation, the Cys is optionally turned back into native Ala by desulfurization.

For example, the process described herein can be performed wherein the ubiquitin monomer is prepared of two ubiquitin segments by NCL, such that the fragment attached to the substrate peptide is:

AGKQLEDGRTLSDYNIQKESTLHLVLRLRGG (Ub46-76) and the second fragment being in its thioester form is LQIFVKTLTGKTITLEVEPSDTIENVKAKIQDKEGIPPDQQRLIF (Ub1-45)-SR, whereas the A₄₆ amino acid is temporarily replaced by Cysteine.

R is either hydrogen or a thiol protecting group.

Another option for preparing the Ub of two ubiquitin segments is wherein the fragment attached to the substrate peptide is:

AKIQDKEGIPPDQQRLIFAGKQLEDGRTLSDYNIQKESTLHLVLRLRGG (Ub28-76) and the second fragment, being in its thioester form, is LQIFVKTLTGKTITLEVEPSDTIENVK (Ub1-27)-SR, whereas the A₂₈ amino acid is temporarily replaced by Cysteine.

The ubiquitin can further be ligated from three fragments. In this case, the process described herein needs to be somewhat modified as follows:

First, the segment (Ub46-76) (AKIQDKEGIPPDQQRLIF) is attached to the substrate peptide, as explained hereinabove, via the Lysine side residue.

Separately, the fragments (Ub28-45)-LTF and Ub(1-27)-SR (AGKQLEDGRTLSDYNIQKESTLHLVLRLRGG and (LQIFVKTLTGKTITLEVEPSDTIENVK, respectively) are separately prepared on solid supports, and are thereafter removed from the support and ligated according to the procedure described in PCT/IL2011/000138 entitled “Chemical Preparation of Ubiquitin Thioesters and Modifications Thereof”, which is incorporated by reference as if fully set forth herein, to obtain the Ub(1-45) fragment still attached to the LTF group. Following activation under acidic conditions and a reaction with an external thiol, the Ub1-45 thioester is obtained.

Finally, NCL OF C46-Ub(47-76)-substrate peptide and (UB 1-45)-SR follows, as conducted in the ligation of the two Ub fragments.

Therefore, according to preferred embodiments of the present invention, in step ii of the process described hereinabove, while elongating the ubiquitin fragment by SPPS, a Cys amino acid is introduced as the m₁ amino acid, thereby forming a modified ubiquitin peptide fragment containing m₁ amino acids and having a C-terminal Cys amino acid residue.

Furthermore, in order to enable this Cys amino acid residue to attach to another ubiquitin fragment, the Cys amino acid residue needs to be protected by a suitable protecting group, and therefore this Cys amino acid residue has the general formula II:

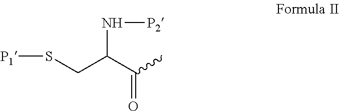

Wherein at least one of the P₁′ or P₂′ on the Cys amino acid residue is selected from: thiazolidine (THz), photolabile 2-nitro benzene, and methylsulfonylethoxycarbonyl (MSC).

The term “amino acid” as used herein includes all natural and non-natural amino acids.

Throughout this specification the natural amino acid residues will be denoted by the three-letter abbreviation or single-letter codes as follows:

| | Three-letter | One-letter |
|---|---|---|
| Amino Acid | abbreviation | abbreviation |
| name | Symbol | Symbol |
| Alanine | Ala | A |
| Arginine | Arg | R |
| Asparagine | Asn | N |
| Aspartic Acid | Asp | D |
| Cysteine | Cys | C |
| Glutamine | Gln | Q |
| Glutamic Acid | Glu | E |
| Glycine | Gly | G |
| Histidine | His | H |
| Isoleucine | Ile | I |
| Leucine | Leu | L |
| Lysine | Lys | K |
| Methionine | Met | M |
| Phenylalanine | Phe | F |
| Proline | Pro | P |
| Serine | Ser | S |
| Threonine | Thr | T |
| Tryptophan | Trp | W |
| Tyrosine | Tyr | Y |
| Valine | Val | V |

Amino acid substitutions are typically of single residues; insertions usually will be on the order of from about 1 to 20 amino acids, although considerably larger insertions may be tolerated. Deletions range from about 1 to about 20 residues, although in some cases deletions may be much larger.

Substitutions, deletions, insertions or any combination thereof may be used to arrive at a final derivative. Generally these changes are done on a few amino acids to minimize the alteration of the molecule. However, larger changes may be tolerated in certain circumstances.

The term “modified amino acid” as used herein is used interchangeably with the term “unnatural amino acid”, and refers to any amino acid and/or amino acid analogue, that is not one of the 20 common naturally occurring amino acids or the rare naturally occurring amino acids e.g., selenocysteine or pyrrolysine. This terms also includes any amino acid that is labeled, marked or protected.

The term “modified Lys amino acid” as used herein refers to a Lys amino acid that is orthogonally protected by a group OP, thereby creating a branching point in this location on the main chain of the substrate peptide.

The term “branching point”, as used herein, refers to a Lys residue on the target peptide, which has been modified with an orthogonal protection group that can be selectively removed during synthesis, thereby forming an open isopeptide linkage pathway on this Lys residue. Thereafter, the Ubiquitin attaches to the main peptide backbone at this location, which is equivalent to the natural isopeptide bond between the ubiquitin and the substrate peptide.

The term “regular protecing group suitable for either Fmoc SPPS or Boc SPPS” includes all SPPS commonly-accepted protecting groups. In general, the term “protecting group” refers to a group that blocks an organic functional group and which can be eliminated under controlled conditions. Protecting groups, their relative reactivities and the conditions under which they remain inert are known to an expert on the subject.

The term “terminal-amine” protecting group refers to regular N-protecting groups, as used in SPPS chemistry. Representative examples of protecting groups for the amino group include, but are not limited to, amides, such as amide acetate, amide benzoate, amide pivalate; carbamates such as benzyloxycarbonyl (Cbz or Z), 2-chlorobenzyl (CIZ) para-nitrobenzyloxycarbonyl (pNZ), te/Y-butyloxycarbonyl (Boc), 2,2,2-trichloroethoxycarbonyl (Troc), (Teoc), 2-(trimethylsilyl)ethyloxycarbonyl (Fmoc) 9-fluorenylmethyloxycarbonyl or allyloxycarbonyl (Alloc), Trityl (Trt), methoxytrityl (Mtt), 2,4-dinitrophenyl (Dnp), &#923;/-[1-(4,4-dimethyl-2,6-dioxocyclohex-1-ylidene)ethyl (Dde), 1-(4,4-dimethyl-2,6-dioxo-cyclohexylidene)-3-methyl-butyl (ivDde), 1-(1-adamantyl)-1-methylethoxy-carbonyl (Adpoc). Most preferably, these groups are Boc or Fmoc.

The term “Orthogonal protecting group” OP, as used herein refers to a protecting group on the amino function of the lysine side chain (ε-amine), that is different than the protecting group on the N-terminal of the Lysine as well as of other amines on the main chain. Therefore, this group can be selectively removed at the required synthetic stage, without being affected by the deprotection of the “regular” protection groups.

According to preferred embodiments of the present invention, the Orthogonal protecting group OP depends on the type of SPPS.

For example, in Fmoc-SPPS, the OP group can be any protecting group that is orthogonal to Fmoc-SPPS, namely:
a) A group that is stable to piperidine (highly basic conditions), which is used to remove the Fmoc protecting group along the synthesis;
b) A group that is stable under coupling conditions of each amino acid; and
c) The removal of such protecting group should not deprotect any of the side chains protecting group (P) nor remove the peptide from the resin.

Examples of OP protecting groups that are suitable for Fmoc-SPPS include, but are not limited to, azide, allyloxycarbonyl (Alloc), 1-[4,4-dimethyl-2,6-dioxo-cyclohexylidene]-3-methylbutyl (IvDde) or 1,1-dichloro-2,2-bis(p-chlorophenyl)ethylene (Dde).

On the other hand, for Boc-SPPS, the OP group can be any protecting group that is orthogonal to Boc-SPPS, namely:
a) A group that is stable to the TFA cycles that are used to remove the Boc group during SPPS;
b) A group that is stable under coupling conditions of each amino acid; and
c) The removal of the orthogonal protecting group to allow on resin isopeptide formation should not affect the side chain protecting groups on the amino acids and remove the peptide from the resin.

Examples of OP protecting groups suitable for Boc-SPPS include, but are not limited to, Fmoc, azide, alloc and IvDde.

The term “selectively removing the OP protecting group” refers to supplying a suitable removing agent, able to remove only the desired OP protecting group, while not affecting any other protecting groups on the peptide.

Exemplary removing agents can be Pd⁰ for removing the alloc protection, tris(2-carboxyethyl)phosphine (TCEP) or Triphenylphosphine (P(Ph)₃) for removing the azide protection, piperidine for removing the FMOC protection, and hydrazine for removing the Dde or ivDde protection.

Additional removing agents are known to a person skilled in the art.

According to preferred embodiments of the invention, the synthesis is an Fmoc synthesis and the orthogonally protecting group is 1-[4,4-dimethyl-2,6-dioxo-cyclohexylidene]-3-methylbutyl (ivDde) as this group is more stable under the conditions of Fmoc removal.

According to further preferred embodiments of the invention, this group is then removed by hydrazine, in particular by using 5% hydrazine in DMF.

The term “Chemically synthesizing” refers to the fact that the obtaining of any of the polypeptide, and in particular obtaining the Ubiquitinated substrate peptide, and of the ubiquitin fragment peptide and the ubiquitin thioester, is not conducted enzymatically or by gene expression, neither in vivo nor in vitro.

The term “at least partially prepared by SPPS” means that at least part of the ubiquitin is formed by the SPPS of amino acid building tools.

The term “further elongated” or “further elongating” includes both SPPS elongation, as well as elongation by NCL.

As shown in the examples given below, after obtaining the initial OP-protected-Lys-branched peptide fragment, a number of different combinations of SPPS and NCL can be conducted to obtain the desired ubiquinated peptide conjugates:

Initially,

i) the peptide can be further elongated at its N-terminus, beyond the branching point, by SPPS or by NCL through a suitable terminal protecting group, to be followed by removal of the OP group on the Lys and attachment of a first ubiquitin fragment in the unprotected Lys side chain; OR

ii) the OP group on the Lys can be removed, to allow the attachment of a first ubiquitin fragment in the unprotected Lys side chain, to be followed by further elongation of the peptide at its N-terminus, beyond the branching point, by SPPS or by NCL through a suitable terminal protecting group.

Then, both the ubiquitin fragment and the peptide may be further elongated by SPPS or NCL, taking into consideration the inherent limitations of the SPPS method, namely the limited number of amino acids which can be efficiently added via SPPPS.

Once SPPS is terminated, cleavage from the resin is conducted to obtain the free ubiquinated peptide conjugates of the present invention, which may then be further elongated by Native Chemical Ligation.

The following conditions can be used for cleavage of the peptide-LTF to release the assembled polypeptide from the solid phase using TFA/TIS/H₂O (95:2.5:2.5).

The term “free ubiquinated peptide conjugate” as used herein refers to a free ubiquinated peptide conjugate that has been cleaved from the solid support and is no longer attached thereto. The polypeptide fragment obtained by SPPS is an unprotected polypeptide or fragment. Namely, it does not contain protection groups on the side chains of the amino acids.

Scheme 4A exemplifies the synthetic route of the invention for a number of peptides whereas the orthogonally protected Lys, Fmoc-Lys-(ivDde)-OH, was used to introduce the isopeptide in a site specific manner.

The obtained peptide has the amines in the Lys amino acid protected by two different groups: the ε-amine of the Lys was protected orthogonally by a ivDde protecting group, whereas the N-terminus amine was protected by an Boc group.

As shown in Scheme 4A and exemplified in Example 2, the ε-amine (side-amine) of the Lys residue is unmasked selectively by applying 5% hydrazine in DMF, and can then followed by additional Fmoc-SPPS of the Ub segment (such as Ub(₄₆₋₇₆)).

The Ub-peptide conjugates and fragments thereof that were prepared according to this scheme are presented in Table 1:

TABLE 1

| | | Ub-fragment- | Full Ub- |
|---|---|---|---|
| Peptide I | Pre-1 | peptide 1 | peptide 4 |
| LYKAG | Compound 5 | Compound 11 | Compound 17 |
| FKTEG | Compound 6 | Compound 12 | Compound 18 |
| LIFAGKQLE | Compound 7 | Compound 13 | Compound 19 |
| AVTKYTSK | Compound 8 | Compound 14 | Compound 20 |

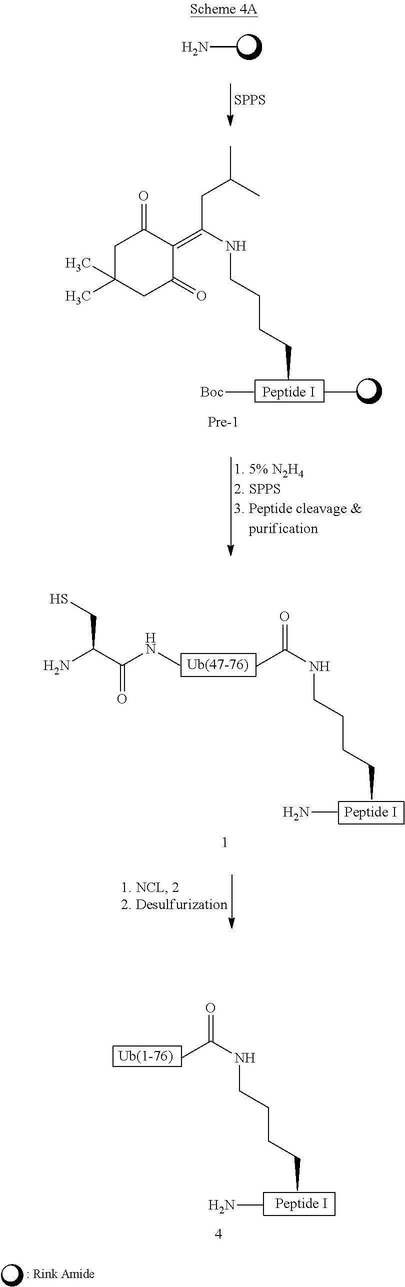

Thus, using the strategy outlined above, Ub-fragment-peptides 11-14 (having general structure 1) were efficiently synthesized as indicated by the crude peptide analysis using HPLC and mass spectrometry in 25-30% yield.

Separately, Ub(₁₋₄₅)-thioester 2, was synthesized as was previously described by the present inventors (see PCT/IL2011/000138), by applying Fmoc-SPPS and the N-acyl urea based chemistry.

The ligation between Ub-thioester 2 and any of the peptides 11-14 was carried out under NCL conditions i.e. 6 M Gn.HCl, 200 mM phosphate buffer, pH 7.5 in the presence of 2% (v/v) thiophenol/benzylmercaptan.

The purified ligated product was subjected to metal free desulfurization conditions in order to turn any un-natural Cysteine into an Alanine amino acid.

Thus, according to preferred embodiments of the present invention, the process described herein optionally further comprises desulfurizing the ubiquinated peptide conjugate to convert any unnatural Cys amino-acids into the respective native Ala amino acids.

The natural structure of the desulfurized product 17 was confirmed upon treatment with UCH-L3, whereas after 3 hours a complete hydrolysis was achieved affording both the hydrolyzed Ub-COOH and the LYKAG peptide.

The same strategy was applied for the rapid synthesis of the ubiquitinated peptides 18-20.

The present approach, through further elaboration, enables to increase the length of the ubiquitinated peptide at the N- or C-termini.

This is done by choosing a terminal protection group on the peptide, that can be selectively removed upon completeing the synthesis of the Ub on the Lysine residue of the peptide.

This protecting group, P′ (such as P1′ and P2′) is selected from thiazolidine (THz) (which can protect both the thiol group of the Cys and its amine group), photolabile 2-nitro benzene (which can protect the thiol group of the Cys) and methylsulfonylethoxycarbonyl (MSC) (which can protect the amine group of the Cys).

Then, a second peptide or peptide fragment, which is activated as a thioester or thioester-analogue, such as NBz, is reacted through NCL with the unmasked Ub-peptide.

An exemplary process for the elongation of peptides, is shown in Scheme 4B, whereas the side protection group on the peptide is Thz, and the second peptide is activated by Nbz.

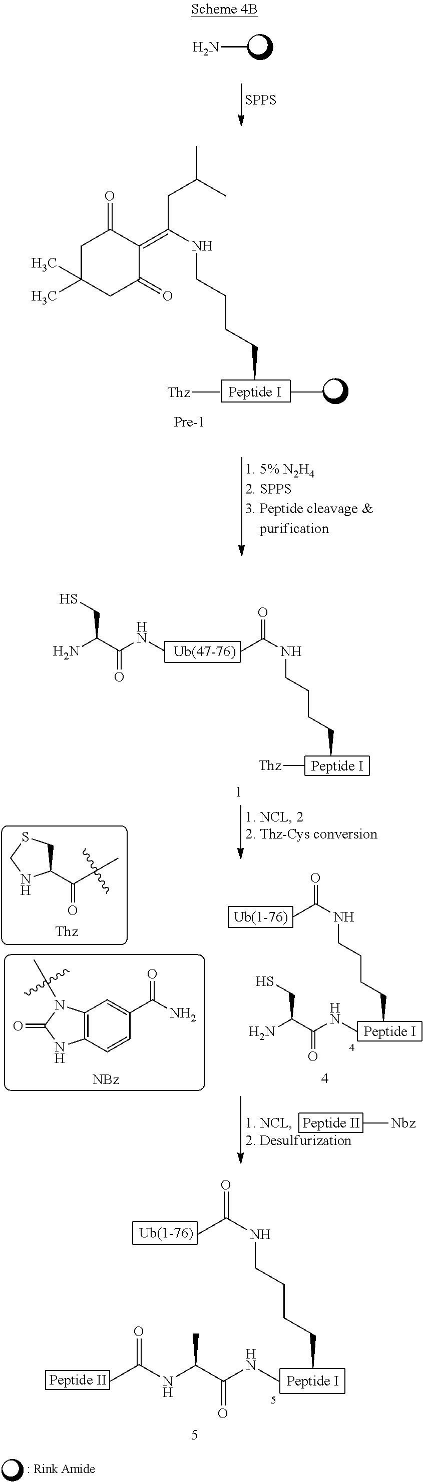

The Ub-peptide conjugates and fragments thereof that were according to this scheme are presented in Table 2:

TABLE 2

| | | | | | Full Ub- |
|---|---|---|---|---|---|
| | | Ub-fragment- | Full Ub- | | elongated |
| Peptide I | Pre-1 | peptide 1 | peptide 4 | Peptide II | peptide 5 |
| LYKAG | Compound 9 | Compound 15 | Compound 29a | LYRAG | Compound 21 |
| VTKYTSK | Compound 10 | Compound 16 | Compound 29b | AVSEGTK | Compound 22 |
| | | | | GELAKHA | Compound 23 |
| | | | | VSEGTK | |
| | | | | IQTAVRL | Compound 24 |
| | | | | LLPGELA | |
| | | | | KHAVSEG | |
| | | | | TK | |

For example, to expand the length of the backbone LYKAG peptide at the N-terminus, ubiquinated peptide conjugate 15 was prepared from pre-peptide 9 (Thz-LYK(ivDde)AG). Subsequent to the first ligation, the Thz was converted fully to Cys using methoxylamine (Bang, D. et al. (2005). Angew. Chem. Int. Ed. 44, 3852-3856) to allow for sequential backbone ligation with LYRAG-thioester-Nbz, after which a desulfurization step gave the ubiquitinated peptide 21 in 25% isolated yield over two steps.

The method depicted in Scheme 4B enables the chemical synthesis of large proteins, thereby overcoming the limitations of SPPS.

For example, the present inventors have successfully synthesized ubiquitinated H2B peptide, which has 125 amino acids, as shown in Scheme 5 using the key full ubiquitinated peptide H2B(₁₁₈₋₁₂₅) intermediate (compound 27 in Scheme 5), which is the equivalent of peptide 1 in Scheme 4B. Ub(A46C) signifies a full Ub peptide, whereas the Cys amino acid in position 46 is unnatural Arg.

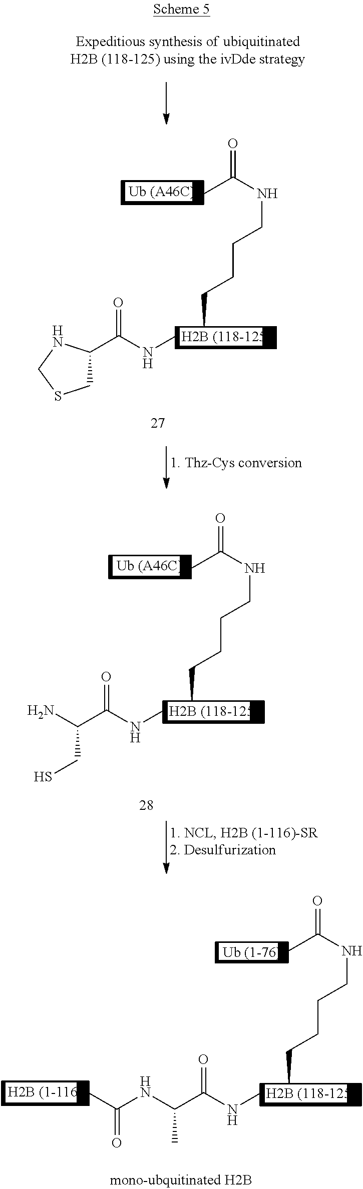

The sequence of the H2B is shown below, whereas the ligation site (A) is underlined and K* corresponds to the ubiquitination site:

¹PEPAKSAPAPKKGSKKAVTKAQKKDGKKRKRSRKESYSVYVYKVLKQVHPDTGISS
KAMGIMNSFVNDIFERIAGEASRLAHYNKRSTITSREIQTAVRLLLPGELAKHAVSEGTK
AVTK*YTSSK125

As seen in Scheme 5, peptide 27 was prepared according to preferred embodiments of the present invention by the method shown in Scheme 4B. This peptide was isolated in 35% yield. The pseudoproline derivative of Tyr₁₂₁Thr₁₂₂, was optionally added during SPPS to improve the synthesis by preventing any aggregation on the resin. This group was removed in the TFA cleavage stage to yield the Tyr-Thr junction.

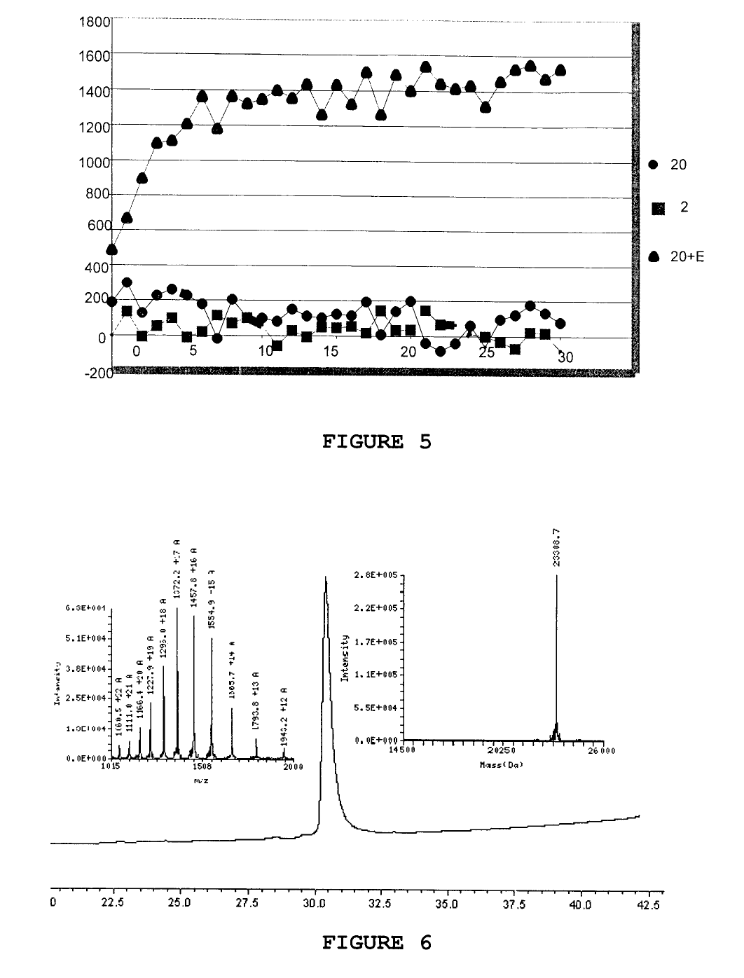

Ligation of compound 27 with the Ub(₁₋₄₅)-thioester 2, followed by Thz to Cys conversion gave the desired building block 28, in 31% yield. Conversion of this key ubiquitinated peptide to the full length H2B was done by NCL with H2B(1-116)-SR. The unnatural Ub(A46C) is optionally turned to the native Ub(1-76) in the final step by desulfurization. FIG. 6 shows the HPLC and mass spectrometry analysis data of the ubiquitinated H2B with the expected mass of 23308.7.

It should be noted that the elongation of the backbone peptide can proceed either through its N-terminal, as shown in Scheme 4B and in Scheme 5, or through its C-terminal, as shown in Scheme 4C, by equipping the C-terminus of the backbone peptide with a N—S acyl transfer device, which can be activated after the first ligation step furnishing the thioester functionality. Alternatively, a kinetically controlled ligation can be used to increase the length of the ubiquitinated peptide at the C-terminus. In this case, the backbone peptide would bear a less reactive alkylthioester compared to the thioester of peptide 2.

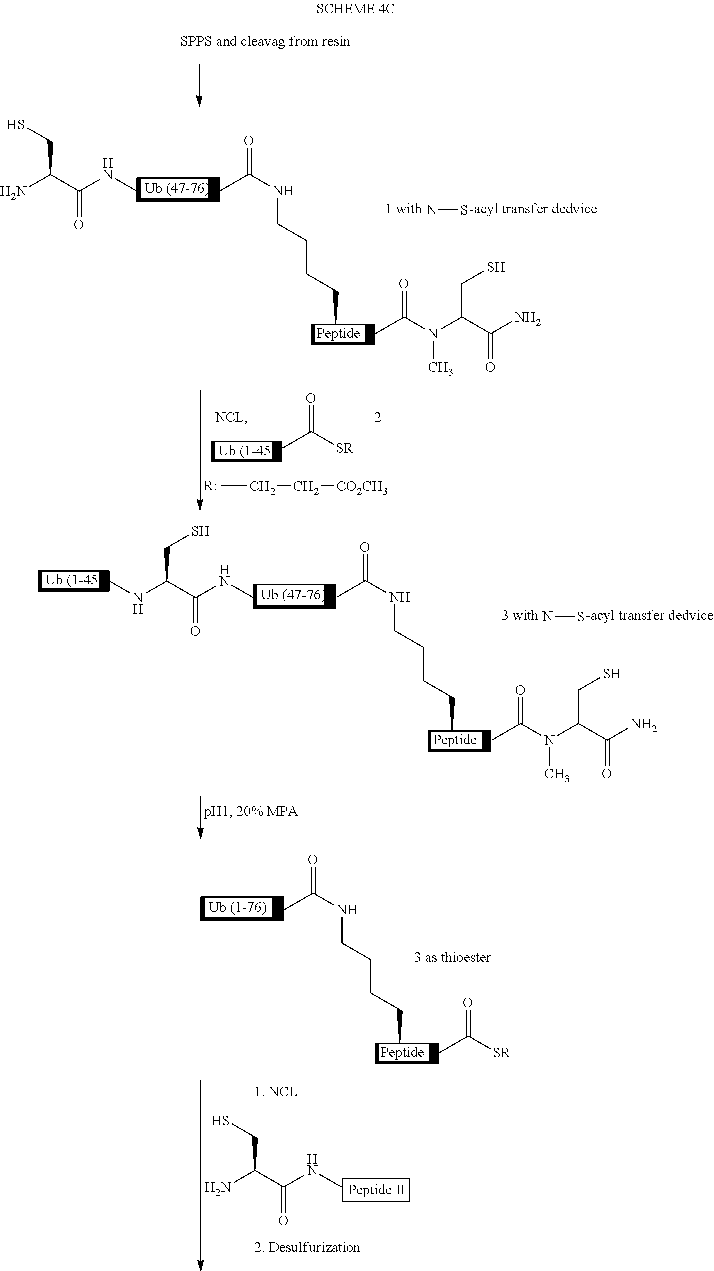

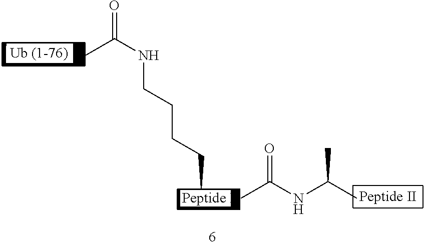

The term “N-acyl transfer device” used interchangeably with the terms “Latent thioester Functionality”, “LTF”, “thioester device” or “switchable device”, describes any functionality that is able to undergo a S->N acyl transfer and withstand the removal from the solid support, as well as the ligation conditions. This functionality therefore serves to introduce into the polypeptide structure, a precursor to a thioester group to be unmasked at later stages of the reaction, only upon an activation step, upon providing acidic conditions.

Preferably, the “Latent thioester Functionality” has the general structure outlined in Formula III:

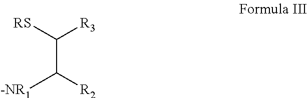

Wherein:

R is either hydrogen or a thiol protecting group;

The compound of formula III would attach to the growing peptide through the Nitrogen attached to R₁, whereas:

R₁ is selected from the group consisting of: hydrogen, C1-C3 alkyl, C1-C3 alkyl-COON, C1-C3 alkyl-CONH₂, C1-C3 alkylene-CONH₂, C1-C3 alkylene-CO₂H, SO₂-alkyl; SO₂-alkyl-CONH₂, benzyl and derivatives thereof, alkyl-nitrile and alkyl-halogens. Additional or specific examples include: iodomethyl, nitromethyl, derivatives of benzyl like o-nitro-benzyl, p-nitro benzyl;

Preferably, R₁ is selected from hydrogen, C1-C3 alkyl, C1-C3 alkyl-CONH₂, SO₂—C1-C3 alkyl-CONH₂, C1-C3 alkyl-COON.

More preferably, R₁ is selected from hydrogen, methyl, ethyl, C1-CONH₂ and C1-COOH.

R₂ and R₃ are selected from the group consisting of: hydrogen, CO₂H, CH₂CO₂H, —CH₂OH, CONH₂, CH₂—CONH₂ and CH₂NH₂, as well as N-protected derivatives thereof.

Preferably, R₂ is selected from hydrogen, CONH₂ and N-protected derivatives thereof. This includes for example CO—N(prolyne amino acid).

According to one specific embodiment of the invention, R₃ is hydrogen.

Furthermore, at least one of R₁ and R₂ should contain a linking group CONH₂ or N-protected derivatives thereof, that would be attached to the solid support.

As noted herein, optionally, the thiol side chain (R) in this latent thioester functionality is protected to avoid intramolecular N—S acyl transfer in the cleavage step from the SPPS resin.

Examples of thiol-protecting groups include, but are not limited to, triphenylmethyl (trityl, Trt), acetamidomethyl (Acm), benzamidomethyl, 1-ethoxyethyl, acetyl, benzoyl, substituted and unsubstituted benzyl groups and the like.

Preferably, the thiol-protecting group is a substituted benzyl group, whereas the phenyl group is substituted by an alkoxy, such as methoxy, ethoxy and the like or by a nitro group.

Most preferably, the thiol protecting group is a photo-labile thiol group, such as 2-nitrobenzyl.

In order to conduct additional ligations on the C-terminal of the ubiquinated peptide conjugate, as shown in Scheme 4C, the thiol-protecting group, if present, is removed (for example by UV), followed by treatment with a thiol, such as MPA, under acidic conditions (i.e. pH<4), to afford the target polypeptide-thioester, that can then further undergo NCL with an additional peptide fragment having a Cys N-terminal amino acid, or an equivalent thereof.

According to some preferred embodiments of the invention, exemplified below, the Latent Thioester Functionality (LTF) is selected from:
i. R=hydrogen or 2-nitrobenzyl; R₁=hydrogen or methyl; R₂=CONH₂; R₃=hydrogen;
ii. R=hydrogen or 2-nitrobenzyl; R₁=hydrogen or methyl; R₂=CO—N-pyrroline; R₃=hydrogen;
iii. R=hydrogen or 2-nitrobenzyl; R₁=methyl, ethyl or benzyl; R₂=hydrogen; R₃=hydrogen;
iv. R=hydrogen or 2-nitrobenzyl; R₁=C1 alkyl-CONH₂ or C1 alkyl-COON; R₂=hydrogen; R₃=hydrogen.

Most preferably, the Latent Thioester Functionality (LTF) is N-methyl cysteine. In this case, R₁ is methyl; R₂ is CONH₂ and R₃ is hydrogen. The inventors have shown that the N-methyl cysteine reacts as expected, both when R is hydrogen and both when it is 2-nitrobenzyl.

It can be seen that the “latent thioester functionality” attached to the C-terminal side of a peptide or a fragment thereof (for example as depicted by Formula II) is independent and stable and can be kept as such until the moment when ligation and/or activation are required, the LTF group acting as a “switchable device”.

When the LTF is the amino acid being attached to the solid support, there is obtained, after cleavage of the ubiquinated peptide conjugate or ubiquinated peptide conjugate fragment from the solid support in step iii of the process described hereinabove, the ubiquinated peptide conjugate having a “latent thioester functionality” on its C-terminal side, as generally depicted in Formula IV below:

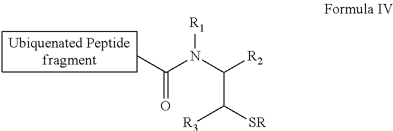

wherein R, R₁, R₂ and R₃ are as defined hereinabove.

Once this fragment is ligated with another peptide fragment containing a Cys N-terminal amino acid, the ubiquitinated peptide conjugate obtained in the previous steps is thereby further elongated in its C-terminal.

Examples of some protected latent thioester functionality attached to peptides, and their reactions to obtain the polypeptide-thioesters, are shown in scheme 5B below:

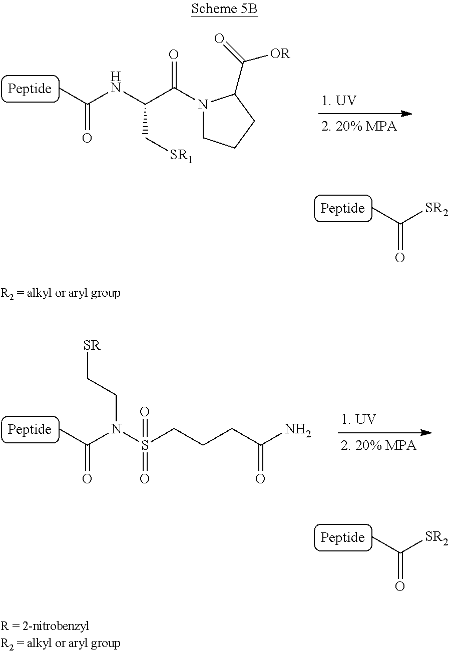

According to preferred embodiments of the present invention, the elongated peptides described hereinabove can be further elongated as indicated above, by initially including a P′ protecting group on the peptide I, that is removed at a later stage to be further ligated with a third peptide III. The same can be done, by starting from the process of Scheme 4B and further elongating by an N—S acyl transfer device is shown in Scheme 4C.

Furthermore, the present invention allows a large degree of flexibility in the synthesis, in the sense that the SPPS and NCL can be combined in any number of ways, taking into consideration the overall limitation of SPPS to efficiently add up to about 80 amino acids, preferably up to about 70 amino acids, and more preferably up to about 50-70 amino acids.

For example, the Ub-H2B protein can be prepared by growing part I of the H2B, containing the branching point Lys (K*) amino acid, on the solid support by SPPS, then adding to it the complimentary part of the H2B, while keeping the orthogonally-protected Lys in its protected state, to be followed by unmasking of the OP group, and growing of the Ub to a certain extent (as dictated by the length of peptide synthesized by SPPS in the first stage), and finally ligating this fragment with a Ub-thioester complimentary fragment.

Alternatively, the Ub-fragment-H2B peptide fragment can be prepared as shown in Scheme 4B and Scheme 5, then the complimentary H2B peptide fragment is added by NCL, and only at that stage is the Ub-thioester or Ub-Nbz added to provide the final full-Ub-prolonged peptide.

As can be seen in the processes described above, as well as in the examples section below, and in FIG. 1 hereinabove, the present inventors have developed a synthetic route enabling to obtain a variety of Ubiquinated peptide conjugates, linked via an isopeptide bond, whereas the ubiquitin residue is either a full-chain Ub, or a fragment thereof, and whereas the substrate or target protein, to which this Ub is attached, can have a varying number of amino acids, and may even be the ligation product of several peptides.

Thus, according to another aspect of the invention, there is now provided a ubiquinated peptide conjugate comprising a ubiquitin peptide residue UR, attached at its C-terminus to a substrate peptide via a native isopeptide bond, said ubiquinated peptide conjugate having the general structure of Formula I:

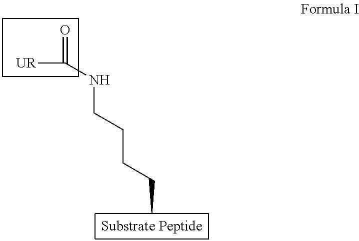

wherein the UR is selected from:
a) a fragment of a ubiquitin peptide;
b) a full-length modified ubiquitin peptide;
c) a fragment of a modified ubiquitin peptide.

As can be seen in Examples, a variety of Ubiquinated peptide conjugates were prepared from a ubiquitin fragment peptide UR containing a Cys C-terminal amino acid that can be attached by NCL to a complimentary ubiquitin fragment.

Therefore, according to preferred embodiments of the invention, the UR ubiquitin peptide fragment has a Cys N-terminal amino acid residue, this Cys amino acid having the general formula II:

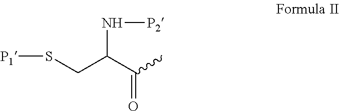

Wherein at least one of the P₁′ or P₂′ on the Cys amino acid residue is selected from: hydrogen, thiazolidine (THz), photolabile 2-nitro benzene, and methylsulfonylethoxycarbonyl (MSC).

As noted hereinabove, the present method should also enable a rapid preparation of deubiquitinases (DUBs) substrate libraries, which could shed light on the unique specificities of particular DUBs. This knowledge would assist in the discovery of specific inhibitors of important DUBs involved in health and diseases.

One such enzyme is the UCH-L3 enzyme, which catalyzes the removal of adducts from the C-terminus of Ub. It is generally accepted that the UCH-L3 preferred substrates are Ub linked to small adducts such as a single amino acid, ethyl ester and short peptides.

So far only one ubiquitinated peptide comprised of 13 amino acids with native isopeptide bond, was enzymatically prepared and indeed found to be a substrate for UCH-L3. The reason that no additional DUB substrates have been prepared is mainly because of the difficulties in preparing highly homogenous ubiquitinated peptides using the E1-E3 enzymatic machinery.

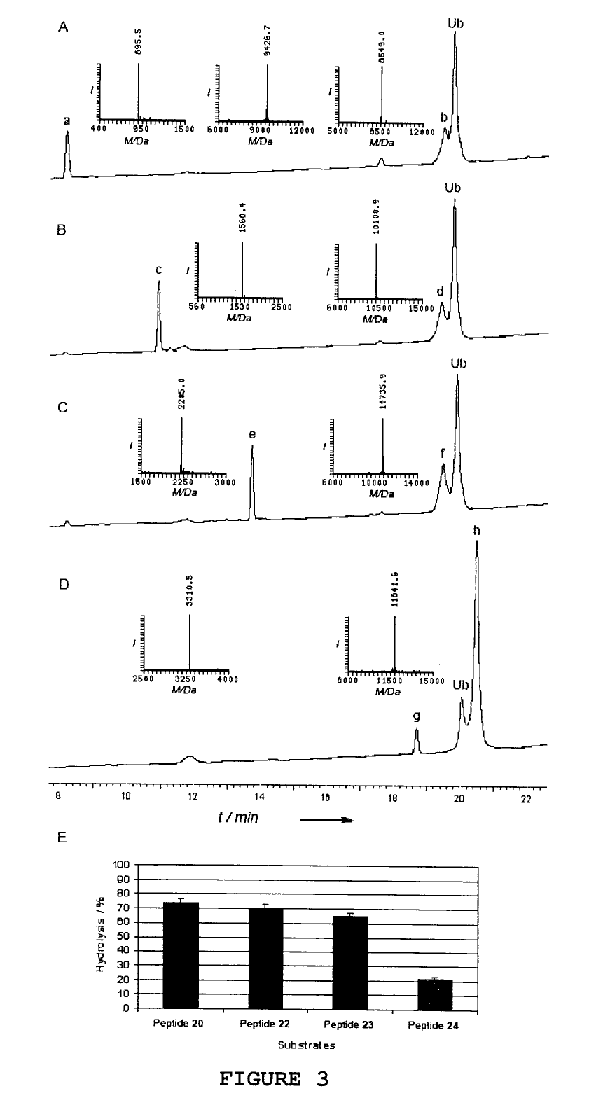

To show the affinity of the Ubiquinated-peptides, prepared according to preferred embodiments of the present invention, four different ubiquitinated peptides 20-24 (Scheme 4B) were assembled, all derived from the C-terminal of H2B, and having various lengths (8, 15, 21 and 31 residues, respectively). These peptides were then tested under the same hydrolysis conditions with UCH-L3. The hydrolysis reaction was monitored by HPLC following the disappearance of the ubiquitinated peptide and the appearance of products, Ub-COOH and the H2B fragment peptide (FIG. 3). As seen in FIG. 3, the peptides 20, 22, and 23 with up to 21 amino acids were cleaved with a similar efficiency and gave a 65-75% hydrolysis within 30 minutes and were completely disassembled within 90 minutes. These results support the notion that UCH-L3 tolerates various peptide sequences.

Furthermore, the newly provided method of preparing Ub-peptides has enabled the developement of potential HTS assays, which could be adopted and modified to any DUBs.

One example, that has been successfully demonstrated by the present inventors is depicted in scheme 6, and is based on a Förster resonance energy transfer (FRET) assay and should maximally represent the natural substrate to allow screening with high sensitivity and accuracy.

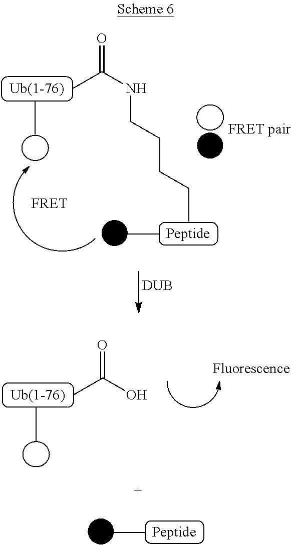

The Peptide in Scheme 6 can be any peptide capable of linking to the Ub, including the Ub peptide itself. In this case, a doubly-labeled di-Ub peptide is obtained.

The proof of concept was done using the following ubiquitinated peptide ((Nle)FKTEG), which was labeled with MCA (7-methoxycoumarin-4-acetic acid), while the ubiquitin was labeled with Dnp (N1-(2,4-dinitrophenyl)ethane-1,2-diamine) as fluorophore as shown in scheme 7 below.

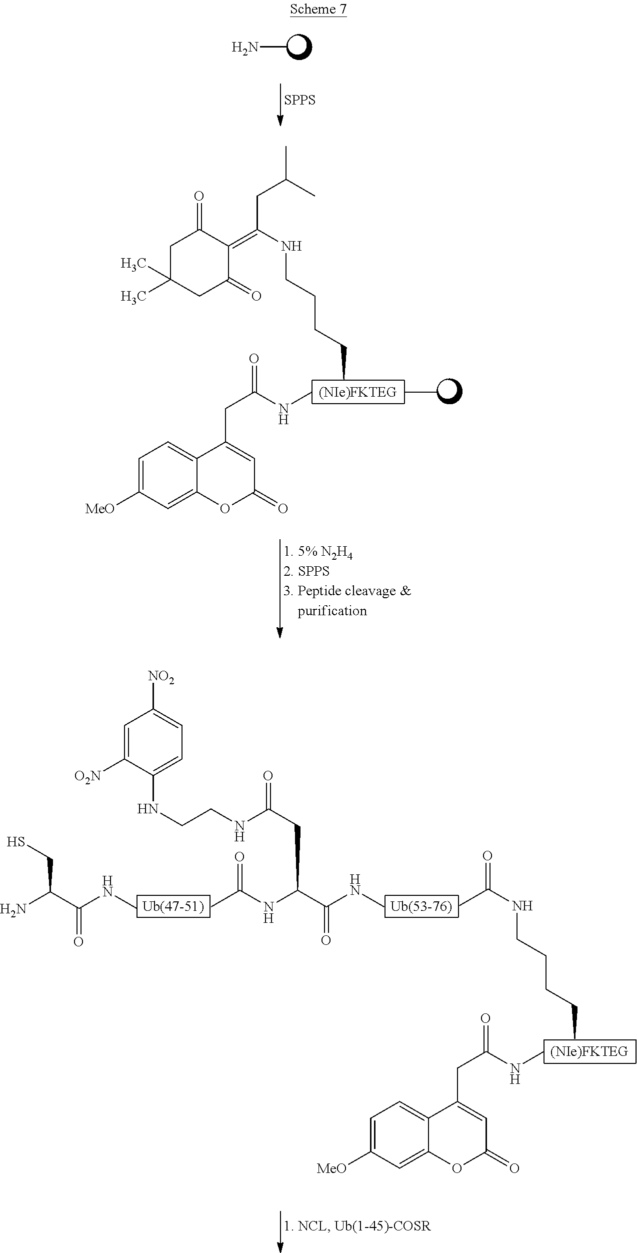

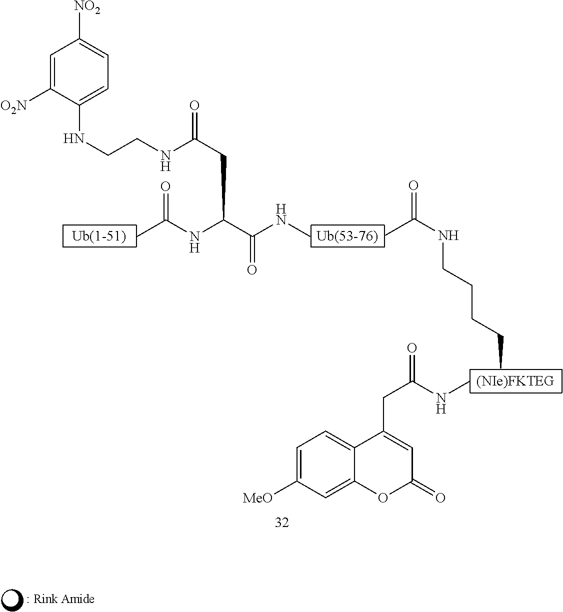

The process shown in Scheme 7 allows for the first time to label the Ub which is attached to the peptide in a highly specific manner, something that is not possible when adding the Ub enzymatically, for example as done in the process of Scheme 1, where NCL of the expressed full Ub is conducted.

Thus, according to preferred embodiments of the present invention, the ubiquitin residue UR contains at least one labeled amino acid. This labeled amino acid is introduced into the ubiquitin sequence during SPPS, as described hereinabove.

The term “labeled amino acid”, as used herein, refers to an amino acid having a detectable label associated therewith or attached thereto. The label may be any suitable labeling substance, including but not limited to a radioisotope, an enzyme, an enzyme cofactor, an enzyme substrate, a dye, a hapten, a chemiluminescent molecule, a fluorescent molecule, a phosphorescent molecule, an electrochemiluminescent molecule or a chromophore.

Similarly, according to another preferred embodiments of the present invention, the substrate peptide may also contain at least one labeled amino acid.

According to yet another preferred embodiment of the present invention, both the ubiquitin residue UR and the substrate peptide contain at least one labeled amino acid.

According to a preferred embodiment of the present invention, the amino acid is labeled by a fluorescent agent.

The term “fluorescent agent” is used interchangeably with the term “fluorophore” and refers to a compound that is inherently fluorescent or demonstrates a change in fluorescence upon binding to a biological compound or metal ion, i.e., fluorogenic.

Numerous fluorophores are known to those skilled in the art and include, but are not limited to, coumarin, cyanine, acridine, anthracene, benzofuran, indole, borapolyazaindacene and xanthenes including fluorescein, rhodamine and rhodol as well as other fluorophores described in the art.

Some preferred fluorescent groups used as labels in the present invention include, but are not limited to 7-methoxycoumarin-4-acetic acid (MCA), and N1-(2,4-dinitrophenyl)ethane-1,2-diamine (Dnp).

An example of such a doubly-labeled ubiquinated peptide conjugate is a FRET pair, wherein the labeling is a fluorescent labeling.

The term “FRET” as used herein is used interchangeably with the terms “fluorescence resonance energy transfer” or “Forster resonance energy transfer”, and refers to the radiation-less transmission of an energy quantum from its site of absorption (the donor) to the site of its utilization (the acceptor) in a molecule, or system of molecules, by resonance interaction between donor and acceptor species, over distances considerably greater than inter-atomic, without substantial conversion to thermal energy, and without the donor and acceptor coming into kinetic collision.

A donor is a moiety that initially absorbs energy (e.g., optical energy or electronic energy).

The term “acceptor” refers to a chemical or biological moiety that accepts energy via resonance energy transfer. In FRET applications, acceptors may re-emit energy transferred from a donor fluorescent or luminescent moiety as fluorescence and are “fluorescent acceptor moieties.” As used herein, such a donor fluorescent or luminescent moiety and an acceptor fluorescent moiety are referred to as a “FRET pair.” Examples of acceptors include coumarins and related fluorophores; xanthenes such as fluoresceins and fluorescein derivatives; fluorescent proteins such as GFP and GFP derivatives; rhodols, rhodamines, and derivatives thereof; resorufins; cyanines; difluoroboradiazaindacenes; and phthalocyanines Acceptors, including fluorescent acceptor moieties, can also be useful as fluorescent probes in fluorescence polarization assays.

Other labeled pairs of interest include, but are not limited to, enzyme/substrate, enzyme/cofactor, luminescent/quencher, luminescent/adduct and dye dimers.

As is demonstrated hereinabove, the process of the present invention provides a tool for preparing ubiquinated peptide conjugates.

One important application of such ubiquinated peptide conjugates is in the preparation of specific binding pair conjugates.

The term “specific binding” refers to that binding which occurs between such paired species as enzyme/substrate, receptor/agonist, antibody/antigen, and lectin/carbohydrate which may be mediated by covalent or non-covalent interactions or a combination of covalent and non-covalent interactions. When the interaction of the such two species produces a non-covalently bound complex, the binding which occurs is typically electrostatic, hydrogen-bonding, or the result of lipophilic interactions. Accordingly, “specific binding” occurs between a paired species where there is interaction between the two which produces a bound complex having the characteristics of an antibody/antigen or enzyme/substrate interaction. In particular, the specific binding is characterized by the binding of one member of a pair to a particular species and to no other species within the family of compounds to which the corresponding member of the binding member belongs. Thus, for example, an antibody typically binds to a single epitope and to no other epitope within the family of proteins.

Thus, according to preferred embodiments of the present invention, the substrate peptide of the present invention has a specific binding affinity with a species selected from an enzyme, an antigen, an agonist, an antibody, a lectin, and a carbohydrate.

In one specific application of interest, the enzyme is a deubiquetenase (Dub) and the substrate peptide is one that is a substrate of a Dub enzyme.

The present inventors have successfully shown that the ubiquinated peptide conjugates of the present invention have a natural structure and therefore have a high affinity for some known Dubs.

Thus, according to another aspect of the invention, there is now provided the use of the ubiquinated peptide conjugates described hereinabove in conducting a High Throughput Screening Assay for detecting Deubiquitenase (DUBs) inhibitors.

The term “High Throughput Screening” (HTS) is used to describe the experimental paradigm than enables the testing of large collections of diverse chemicals against drug targets to identify molecules for entry into chemical optimization programs. A person skilled in the art is in the possession of accumulated knowledge relating to organic molecule library design and maintenance, reagent generation, assay design, laboratory automation and data analysis, fundamental to HTS.

There follows that according to yet another aspect of the present invention, there is now also provided a kit for conducting High Throughput Screening (HTS) assays for identifying potential inhibitors against one or more deubiquitinases (DUBs), this kit comprising:
a) A DUB;
b) A labeled ubiquinated peptide conjugate comprising a ubiquitin peptide residue UR, attached at its C-terminus to a substrate peptide via a native isopeptide bond, said ubiquinated polypeptide conjugate having the general structure of Formula I.

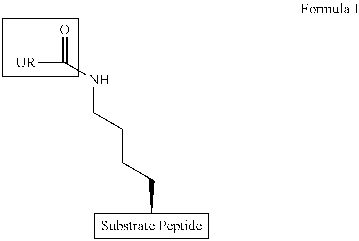

Wherein the UR is a full-length ubiquitin peptide, or a fragment thereof, containing at least one labeled amino acid; and
c) An organic molecule to be tested as a possible inhibitor to this DUB.

According to yet another aspect of the invention, there is now provided the use of the ubiquinated peptide conjugates described hereinabove in the preparation of antigens for developing linkage-specific antibodies.

It should be recognized that any of the binding agents disclosed herein or otherwise known in the art can be reversed. Thus, biotin, e.g., can be incorporated into either a polypeptide or a solid support and, conversely, avidin or other biotin binding moiety would be incorporated into the support or the polypeptide, respectively. Other specific binding pairs contemplated for use herein include, but are not limited to, hormones and their receptors, enzyme, and their substrates, a nucleotide sequence and its complementary sequence, an antibody and the antigen to which it interacts specifically, and other such pairs knows to those skilled in the art.

In summary, the present inventors have successfully shown a novel method for the rapid synthesis of ubiquitinated peptides employing only SPPS and NCL. Using these tools several ubiquitinated peptides were straightforwardly prepared.

One of these peptides is the ubiquitinated C-terminal H2B, which could lead to an efficient synthesis of mono-ubiquitinated H2B. The present method allows for full control of the ubiquitinated peptide, which could aid in studies of different Ub modifications e.g. specific labeling. The present approach has been further shown to enable the rapid assembly of a variety of ubiquitinated peptides for various studies related to Ub biology and to facilitate the studies in unraveling the effect of ubiquitination on histone biology.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

### EXAMPLES

Reference is now made to the following examples, which together with the above descriptions, illustrate the invention in a non limiting fashion.

### Materials and Analytical Methods

Materials:

The meaning of the abbreviations used in the description and the claims is as outlined below:
MCA 7-methoxycoumarin-4-acetic acid
Dnp N1-(2,4-dinitrophenyl)ethane-1,2-diamine
Nle Norleucine (a Methionine analogue)
Dde 1,1-dichloro-2,2-bis(p-chlorophenyl)ethylene
IvDde 1-[4,4-dimethyl-2,6-dioxo-cyclohexylidene]-3-methylbutyl
Alloc allyloxycarbonyl
Fmoc 9-Fluorenylmethoxycarbonyl-
Boc t-Butoxycarbonyl-
Thz thiazolidine
MSC methylsulfonylethoxycarbonyl
Nbz N-acylurea
DIEA Diisopropylethylamine
TFA Trifluoraceticacid
DMF N,N′-Dimethylformamide
HBTU O-Benzotriazole N,N,N′,N′-tetramthyl-uronium-
HOBt 1-Hydroxybenzotriazole
DBU 1,8-Diazabicyclo[5.4.0]undec-7-ene
HATU O-(7-Azabenzotriazol-1-yl)-N,N,N′,N′-tetramethyluronium-hexafluoro-phosphate
MEI Methyl iodide
TBAF Tetrabutylammonium Fluoride
MPA 3-mercaptopropionic acid
DTT Dithiothreitol
Tris Tris-(hydroxymethyl)aminomethane
THF Tetrahydrofuran
NaHMDS Sodium Hexamethyldisilazane

DMF was purchased in biotech grade. Commercial reagents were used without further purification.

Resins, protected and unprotected amino acids, and coupling reagents (HBTU, HOBt) were purchased from Novabiochem.

Buffer B is acetonitrile with 0.1% v/v TFA and buffer A is water with 0.1% v/v TFA.

Methanol, triethylamine, THF, ether were purified and dried before use.

The n-hexane used was the fraction distilling between 40-60° C.

Natural ubiquitin, which was used for comparison (from bovine erythrocytes) was purchased from Sigma.

Ubiquitin thioesters were prepared according to the process described in another patent application of the present inventors: PCT/IL2011/000138 entitled “Chemical Preparation of Ubiquitin Thioesters and Modifications Thereof”, which is incorporated by reference as if fully set forth herein.

In particular, Peptide 2 thioester Ub(1-45)-thioester was prepared according to the process described in Example 2 of PCT/IL2011/000138, in ˜80% crude yield and 35% pure yield.

Furthermore, Nbz derivatives of the thioesters of the peptides AVSEGTK, GELAKHAVSEGTK and IQTAVRLLLPGELAKHAVSEGTK, were synthesized as described for peptide 2, and were obtained in 25-35% yield.

H2B(1-116)-SR was expressed as was shown by Muir, see: McGinty, R. K., Kim, J., Chatterjee, C., Roeder, R. G., Pellois, J.-P., and Muir, T. W. (2008) Chemically ubiquitylated histone H2B stimulates hDot1L-mediated intranucleosomal methylation. Nature 453, 812-816.

All other chemicals were purchased from either Aldrich and/or Fluka.

General Synthetic Methods to Prepare Ubiquitin:

The sequence of the Ub protein is: ¹MQIFVKILTG KTITLEVEPS DTIENVKAKI QDKEGIPPDQ QRLIFAGKQL EDGRTLSDYN IQKESTLHLV LRLRGG⁷⁶.

Ub proteins (termed hereinbelow Ub1, Ub2, Ub3 and Ub4) were prepared from two peptide segments obtained from separate SPPS reactions: an N-terminal fragment containing amino acids 1-45, in its thioester form (this fragment is termed UbN or Ub1N, Ub2N, Ub3N and Ub4N in the text below), and a C-terminal fragment containing amino acids 46-76 (this fragment is termed UbC or Ub1C, Ub2C, Ub3C and Ub4C in the text below). If needed, one or more of the amino acids are modified by a unnatural amino acid during the SPPS stage (for example replacing one or more of the 7 natural lysines with an analogue, such as a protected or non-protected mercaptolysine; or—replacing natural Alanine amino acids by Cysteine amino acids).

The two segments were coupled using native chemical ligation (6M Gn.HCl, pH 7 in presence of 2% thiophenol). Desulfurization was used to turn any un-natural Cysteine into an Alanine amino acid.

0.2 M Methoxylamine was used to unmask the Thz-protected mercaptolysine.

SPPS was carried out manually in syringes, equipped with teflon filters, purchased from Torviq or by using an automated peptide synthesizer (CS336X, CSBIO). If not differently described, all reactions were carried out at room temperature.

All other reactions were carried out in oven-dried glassware under dry argon.

### Experimental Section

Instrumental Data:

Mass spectrometry was conducted using LCQ Fleet Ion Trap (Thermo Scientific).

Analytical HPLC was performed on a Thermo instrument (Spectra System p4000) using an analytical column (Jupiter 5 micron, C18/C4, 300A 150×4.6 mm) and a flow rate of 1.2 mL/min.

Preparative HPLC was performed on a Waters instrument using semi-preparative column (Jupiter 10 micron, C4, 300A, 250×10 mm) and a flow rate of 5 mL/min as well as a preparative column (Jupiter 5 micron, C18/C4, 300A, 250×22.4 mm) and a flow rate of 25 mL/min.

¹H (500 MHz) and ¹³C (125 MHz) NMR spectra were recorded using CDCl₃ as a solvent. Chemical shifts are reported in d units (ppm) with reference to TMS as an internal standard, and J values are given in Hz. ¹H and ¹³C-NMR spectra were recorded on a Bruker AMX-500 MHz spectrometer.

Flash column chromatography was carried out with silica gel (60-100 mesh).

Analytical thin-layer chromatography (TLC) was performed using thin layer chromatography on pre-coated plates (0.25 mm, silica gel 60 F254).

Compound spots were visualized by UV light (254 nm) and were stained with citric ammonium molybdate.

Circular Dichroism (CD) Analysis:

Samples preparation: The diubiquitin analogues were dissolved in 6 M Gn.HCl 200 mM phosphate buffer (pH 7.86) 5% of the total volume and diluted with 50 mM Tris buffer (pH 7.54). The Gn.HCl buffer was then extracted by Amicon® Ultra-0.5 10 KDa MWCO (Millpore). The exact final concentration of each protein solution was determined using Pierce® BCA Protein Assay Kit (Thermo scientific) and diluted to a final concentration of 10 μM.

The CD measurements were carried out on a Jasco-815 CD spectropolarimeter, at 25° C., by using a quartz cell with 1.0 mm path length and 16 second averaging times. The CD signals, which resulted from the buffer were subtracted from the spectrum of each sample. Data was converted to ellipticity (θ in deg*cm2*dmol−1) according to the equation: [θ]molar=θobs/(nlc), where θobs is the CD signal in degrees, n is the number of peptide bonds, 1 is the path length in centimeters, and c is the concentration in decimoles per cm³.

Evaluation of the UCH-L3 Activity

The ubiquitinated peptides were dissolved in 50 mM Tris buffer. The exact final concentration of each protein solution was determined using Pierce® BCA Protein Assay Kit (Thermo scientific) and diluted to a final concentration of 20 μM. Stock solution of recombinant human UCH-L3, (16.6 μM) was prepared by diluting 25 μg of the enzyme in 50 mM Tris buffer, 150 mM NaCl, 12 mM DTT, pH 8.0. The enzymatic assay was initiated by incubating each substrate (20 μM) with UCH-L3 (0.1 μM) for 30 minutes at 37° C. Enzyme activity was detected by analytical HPLC using C4 column, 0-60% B over 35 minutes. Each experiment was repeated three times and averaged for each case. The percent of hydrolysis was determined from the integration ratio of the Ub to remains of ubiquinated peptide conjugate.

### Example 1

### Preparation of ivDde-Protected-Peptides (5-10)

### Synthesis of Peptide 5

Protected peptide 5 (LYK(ivDde)AG), was synthesized manually by SPPS on 0.1 mmol scale.

Amino acids and HOBT/HBTU were used in 4-fold excess of the initial loading of the resin. DIEA was used in 8-fold excess. Fmoc deprotection was achieved by treatment of the resin with 20% piperidine (3×3 minutes). The N-terminal Leu was coupled as Boc-Leu-OH.

Protected peptides 6-10 were similarly prepared.

The structure of protected peptides 5-10 was monitored and confirmed by HPLC.

### Example 2

### Preparation of Ub(46-76)-Peptides 1

### 2a) Synthesis of Ub-Fragment Peptide 11

The synthesis of Ub-fragment peptide 11 was carried out on Rink amide resin (0.59 mmol/g, 0.1 mmol scale) starting from peptide 5 (LYK(ivDde)AG), which was prepared according to Example 1 hereinabove. Subsequently, treatment with 5% N₂H₄(H₂O) in DMF for 20 minutes followed by DMF wash was performed to remove the ivDde protecting group. This step was repeated four times to ensure a complete removal of the ivDde-protecting group. At this stage, the isopeptide was formed by coupling of Fmoc-Gly-OH followed by elongation of the remaining amino acids using peptide synthesizer (CSBIO, CS336X). The peptide synthesis using the peptide synthesizer was carried out in presence of 4 eq of AA, 10 eq of DIEA and 4 eq of HBTU/HOBT of the initial loading of the resin. The coupling was kept for 1 h and Fmoc-deprotection was achieved using 20% piperidine with 5/10/5 min cycles. Fmoc-Asp(OMPe)-OH was used to minimize aspartimide formation.

Cleavage from the resin: A mixture of TFA, triisopropylsilane and water (95:2.5:2.5) was added to the dried peptide-resin and the reaction mixture was shaken for 2 hours at RT. The resin was removed by filtration and was washed with TFA (2×2 mL). To precipitate the peptide, the combined filtrate was added drop-wise to 10-fold volume of cold ether, centrifugation, decanting of ether, followed by dissolution of residue in acetonitrile-water. HPLC purification afforded the corresponding peptide in ˜25% yield (˜100 mg). Analytical HPLC of the crude and pure peptide 11 were carried on C4 analytical column using gradient of 5-50% B over 40 minutes. The desired Ub-fragment peptide 11 was observed as a peak with the observed mass 4033.2 (calculated mass 4033.6 Da). Additional peaks were observed corresponding to Arg deletion peptide with observed mass 3877 Da (peak b) and to Asp deletion peptide with the observed mass 3919 Da (peak c).

Additional Ub-fragment peptides containing an amine as the R group were similarly prepared:

Ub-fragment peptides 12 and 13 were isolated in 25-30% yield.

Ub-fragment peptide 14 was prepared in a 30% yield.

### 2b) Synthesis of Thz N-Terminating Ub-Fragment Peptides 15 and 16

Ub-fragment peptide 15 was synthesized as described for Ub-fragment peptide 11 in which the Boc-Thz-OH was coupled at the N-terminal of the backbone peptide to allow for sequential ligation.

Analytical HPLC of the crude (top) and pure (bottom) peptide 15 was conducted. The analysis was carried on C4 analytical column using gradient of 5-50% B over 40 minutes. Peak a corresponds to the desired Ub-fragment peptide 15 with the observed mass 4148 (calcd mass=4149.6 Da). Peak b and c correspond to unidentified masses that related to deletion byproducts with multiple deletions.

Additionally, Ub-fragment peptide 16, also containing Thz as the R group was similarly prepared in a 35% yield.

### 2c) Synthesis of Ub-Fragment Peptide Ub46-76-{H2B(118-125}

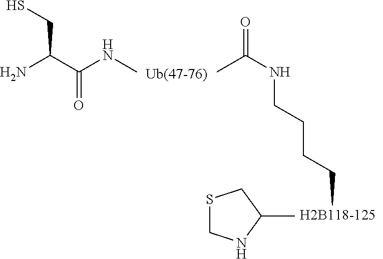

Ub-fragment peptide Ub46-76-{H2B(118-125} was synthesized as described for Ub-fragment peptide 11.

Analytical HPLC of the crude (top) and pure (bottom) Ub-fragment is provided in FIG. 6.

### Example 3

### Native Chemical Ligation of Ub-Fragment Peptide 1 and Ub-Peptide Thioester 2

### 3a) Synthesis of Ubiquitinated Peptide 17

### Ligation:

Peptides 2, (4.0 mg, 1 eq) and 11 (4.03 mg, 1.3 eq), were dissolved in 384 μL of 6 M guanidine.HCl, 200 mM phosphate buffer pH ˜7.0. To this solution 7.7 μL each of benzylmercaptan and thiophenol were added and incubated for 12 hours at 37° C. The reaction was followed using an analytical column and a gradient of 5-25-60% B over 45 minutes. For preparative HPLC a similar gradient was used to afford the ligation product in ˜42% yield (3.0 mg).

### Desulfurization:

The ubiquitinated peptide was dissolved in argon purged 6 M guanidine.HCl 0.2 M Phosphate buffer pH ˜7.0 to a concentration of 2 mM. To this solution, a solution of TCEP (0.5 M) in argon purged guanidine.HCl/phosphate buffer pH 7, 10% (v/v) of t-BuSH and 0.1 M radical initiator VA-044 were added. The mixture was left at 37° C. for 4 hours. The progress of the reaction was analyzed using C4 analytical RP-HPLC employing a gradient of 5-25-60% B over 45 minutes to afford pure desulfurized peptide 17 in 66% yield, (2 mg).

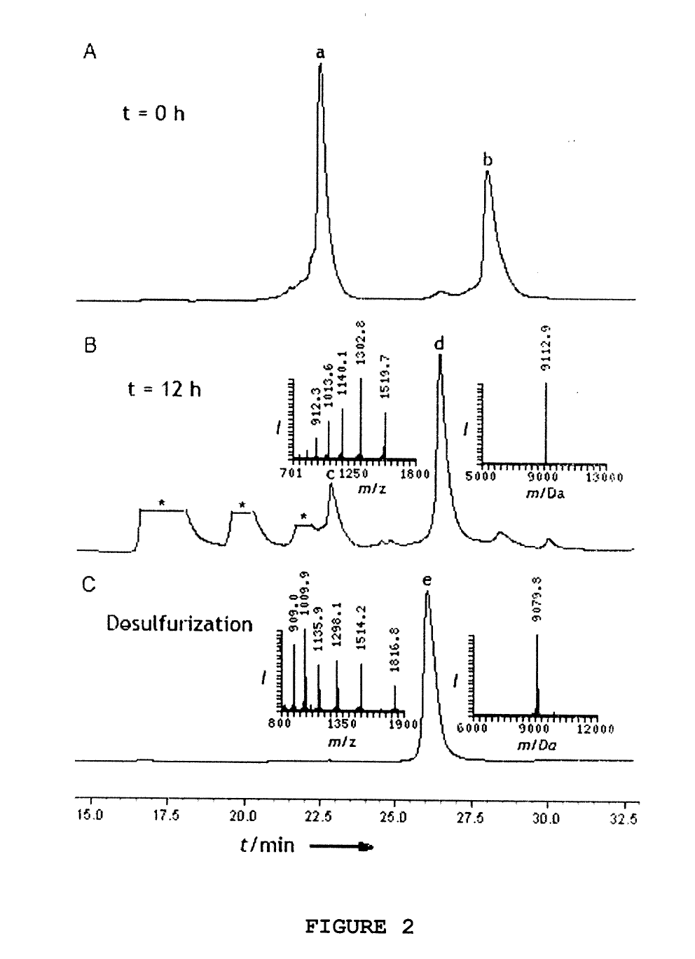

FIG. 2 shows the analytical HPLC trace/(ESIMS) of the ligation reaction in the presence of 2% (v/v) thiophenol/benzylmercaptan. Reported mass is for total protein. FIG. 1A) Ligation at 0 h: Peak a corresponds to peptide 11 with the observed mass of 4034 Da (calcd mass=4033.6 Da). Peak b corresponds to peptide 2 with the observed mass 5198 Da, (calcd mass=5198.9 Da). B) Ligation at 12 h: peak c corresponds to remains of peptide 2, peak d corresponds to the ligation product with the observed mass of 9112.9 Da (calcd mass=9111.5 Da). Ligation was carried out in 6 M Gn.HCl, 200 mM phosphate buffer, pH 7.5 in the presence of 2% (v/v) thiophenol/benzylmercaptan and the product was isolated in 42% yield. C) Desulfurization of the ligation product after 4 h: Peak e corresponds to the desired desulfurized product 17 with the observed mass of 9079.8 Da (calcd mass=9079.5 Da). Peak corresponds to thiol additives.

Enzymatic cleavage with UCH-L3 of peptide 17 was conducted according to the methods described above. After 3 hours a complete hydrolysis was achieved.

### 3b) Synthesis of Ubiquitinated Peptide 18

Ligation:

Peptides 2 (4.65 mg, 1 eq) and 12 (4.4 mg, 1.1 eq), were dissolved in 447 μL of 6 M guanidine.HCl, 200 mM phosphate buffer pH ˜7.5. To this solution 9 μL each of benzylmercaptan and thiophenol were added and incubated for 18 h at 37° C. The reaction was followed using an analytical column and a gradient of 5-25-60% B over 45 min. For preparative HPLC, a similar gradient was used to afford the ligation product in ˜40% yield (3.6 mg).

Desulfurization:

The ubiquitinated peptide (2.2 mg) was dissolved in 6 M guanidine.HCl 0.2 M Phosphate buffer, purged with argon, pH ˜7.0 to a concentration of 2 mM. To this solution, a solution of TCEP (0.5 M) in argon purged guanidine.HCl/phosphate buffer pH 7, 10% (v/v) of t-BuSH and 0.1 M radical initiator VA-044 were added. The mixture was left at 37° C. for 4 h. The progress of the reaction was analyzed using C4 analytical RP-HPLC employing a gradient of 5-60% B over 45 min to afford 1.5 mg pure desulfurized peptide 18 in 67% yield.

Analytical HPLC traces/(ESMS) of the ligation reaction between peptides 2 and 12 after 18 hours show peak c which corresponds to the ligated product with the observed mass of 9142.9 Da (calcd mass=9143.4 Da). Peak d and e corresponds to peptide 2 and its benzylmercaptan exchanged thioester with the observed mass of 5202 Da.

### 3c) Synthesis of Ubiquitinated Peptide 19

Ligation:

Peptides 2 (4.0 mg, 1 eq) and 13 (4.4 mg, 1.25 eq), were dissolved in 384 μL of 6 M guanidine.HCl, 200 mM phosphate buffer pH ˜7.5. To this solution 7.7 μL each of benzylmercaptan and thiophenol were added and incubated for 18 h at 37° C. The reaction was followed using an analytical column and a gradient of 5-25-60% B over 45 min. For preparative HPLC a similar gradient was used to afford the ligation product in ˜37% isolated yield (3.0 mg).

Desulfurization: The ubiquitinated peptide (2.0 mg) was dissolved in argon purged 6 M guanidine.HCl 0.2 M Phosphate buffer pH ˜7.0 to a concentration of 2 mM. To this solution, a solution of TCEP (0.5 M) in argon purged guanidine.HCl /phosphate buffer pH 7, 10% (v/v) of t-BuSH and 0.1 M radical initiator VA-044 were added, sequentially. The mixture was left at 37° C. for 4 h. The extent of the reaction was analyzed using C4 analytical RP-HPLC employing a gradient of 5-25-60% B over 45 min to afford 1.2 mg pure desulfurized peptide 19 (60% yield).

Analytical HPLC traces/(ESMS) of the ligation reaction between peptides 2 and 13 after 18 hours show peak c which corresponds to remains of peptide 13 while peak d corresponds to the ligated product with the observed mass of 9580.2 Da (calcd mass=9580.0 Da). After desulfurization after 10 hours, peak e is observed, which corresponds to peptide 19 with the observed mass of 9548.2 Da (calcd mass=9547.0 Da).

### 3d) Synthesis of Ubiquitinated Peptide 20

This peptide was prepared in similar manner as peptides 17-19, by using the pseudoproline derivative of Tyr₁₂₂Thr₁₂₃, Fmoc-Tyr-Thr(psiMe, Merpro)-OH, which was coupled manually as mentioned above.

Analytical HPLC traces/(ESMS) of the pure peptide 20 showed an observed mass of 9426.7 Da (calcd mass=9425.8 Da).

3e) Evaluation of the UCH-L3 Activity with Peptide 20

The UCH-L3 activity with peptide 20 was evaluated as described in the methods section above.

FIG. 3 depicts Evaluation of the UCH-L3 activity with ubiquitinated peptides 20, 22-24: A) HPLC and mass spectrometry analysis of the enzymatic cleavage of peptide 20 after 30 min. Peak a corresponds to the 8-mer peptide from H2B with the observed mass of 895.5 Da (calcd. 896.0 Da); Peak b is the remaining starting material, peptide 20, with the observed mass of 9426.7 Da (calcd mass=9425.8 Da); the major peak corresponds to Ub-COOH with the observed mass of 8549.0 Da (calcd mass=8548.5 Da). B) HPLC and mass spectrometry analysis of the enzymatic cleavage of peptide 22 after 30 min. Peak c corresponds to the 15-mer peptide from H2B with the observed mass of 1568.4 Da (calcd mass=1568.8 Da); Peak d is the remaining starting material, peptide 22, with the observed mass of 10100.9 Da (calcd mass=10098.7 Da), the major peak corresponds to Ub-COOH. C) HPLC and mass spectrometry analysis of the enzymatic cleavage of peptide 23 after 30 min. Peak e corresponds to the 21-mer peptide from H2B with the observed mass of 2205.0 Da (calcd mass=2204.5 Da); Peak f is remaining starting material, peptide 23, with the observed mass of 10735.9 Da (calcd mass=is 10734.3 Da), the major peak corresponds to Ub-COOH. D) HPLC and mass spectrometry analysis of the enzymatic cleavage of peptide 24 after 30 min. Peak g corresponds to the 31-mer peptide from H2B with the observed mass of 3310.5 Da (calcd mass=3309.9 Da); Peak h is the starting material, peptide 24, with the observed mass of 11841.4 Da (calcd mass=11839.8 Da). E) The percent hydrolysis of ubiquitinated peptides 20, 22-24, which was determined under same reaction conditions. Error bars correspond to the standard deviation of three measurements.

As seen in FIG. 3, the peptides 20 gave a 75% hydrolysis within 30 minutes and was completely disassembled within 90 minutes.

### Example 4

### Native Chemical Ligation to Obtain Prolonged Peptides (Scheme 4B)

4ai) Preparation of Peptide 29a from Peptides 2 and 15

Ligation and Thz-Cys conversion: Peptides 2 (4.0 mg, 1 eq) and 15 (4.15 mg, 1.3 eq), were dissolved in 384 μL of 6 M guanidine.HCl, 200 mM phosphate buffer pH ˜7.0. To this solution 7.7 μL each of benzylmercaptan and thiophenol were added and incubated for 12 hr at 37° C. Subsequently, the mixture was treated with methoxylamine at pH 4 and TCEP (30 eq). The reaction was followed using an analytical column and a gradient of 5-60% B over 45 min. For preparative HPLC a similar gradient was used to afford the ligation product in ˜29% yield (2.5 mg).

4aii) Synthesis of Ubiquitinated Peptide 21

Sequential ligation: the product 29a from the previous step was ligated with LYRAG-thioester-Nbz derivative, as described in step 3 of path B in scheme 4B, to give the ligated product in 44% yield (1.5 mg).

Desulfurization: The ubiquitinated peptide was desulfurized as described above to afford pure peptide 21 in 65% yield (1 mg).

Analytical HPLC traces/(ESMS) of the ligation reaction between peptides 2 and 15 after 18 hours ligation show peak a which corresponds to the ligated product with the observed mass of 9228.3 Da (calcd mass=9226.5 Da), and peak b which corresponds to benzyl thioester derivative of peptide 2 with the observed mass of 5202 Da.

Analytical HPLC traces/(ESMS) of the ligation reaction between peptides 2 and 15 after methoxylamine treatment (after 10 hours) shows the desired product (peak c) with the observed mass of 9216.2 (calcd mass=9214.7 Da).

Analytical HPLC traces/(ESMS) of the ligation reaction between peptides 2 and 15 after sequential ligation with LYRAG-SR (after 12 hours) show peak d which corresponds to the remaining LYRAG-benzyl thioester, peak e corresponds to the desired ligated product with the observed mass of 9776.4 Da (calcd mass=9775.3 Da), and peak which corresponds to thiol additives.

Analytical HPLC traces/(ESMS) of the ligation reaction between peptides 2 and 15 after desulfurization (after 10 hours) show peak f corresponding to peptide 21 with the observed mass of 9712.2 Da (calcd mass=9711.2 Da).

4bi) Preparation of Peptide 29b from Peptides 2 and 16

Ligation and Thz-Cys conversion: Peptide 2, (12.0 mg, 1 eq) and 16 (13.26 mg, 1.3 eq), were dissolved in 1.1 mL of 6 M guanidine.HCl, 200 mM phosphate buffer pH ˜7.5. To this solution 23 μL each of benzylmercaptan and thiophenol were added and incubated for 18 hours at 37° C. Subsequently, the mixture was treated with methoxylamine at pH 4 and TCEP (30 eq). The reaction was followed using an analytical column and a gradient of 5-60% B over 45 min. For preparative HPLC a similar gradient was used to afford the ligation product in ˜31% yield (8 mg).

Analytical HPLC traces/(ESMS) of the ligation reaction between peptides 2 and 16 after 18 hours show peak c which corresponds to remains of peptide 16 while peak d corresponds to the ligated product with the observed mass of 9501.5 Da (calcd mass=9502.0 Da).

Analytical HPLC traces/(ESMS) of the ligation reaction between peptides 2 and 16 after methoxylamine treatment (after 10 hours) shows the desired product (peak e) with the observed mass of 9490.2 (calcd mass=9490.0 Da).

4bii) Synthesis of Ubiquitinated Peptide 22

Sequential Ligation: Peptide 29b (3.0 mg, 1 eq), prepared according to Example 4bi was subjected to sequential ligation with peptide (AVSEGTK)-Nbz (0.8, 3 eq). The peptides were dissolved in 105 μL of 6 M guanidine.HCl, 10 mM TCEP, 200 mM phosphate buffer pH ˜7.2. To this solution 2.1 μL each of benzylmercaptan and thiophenol were added and incubated for 18 h at 37° C. The reaction was followed using an analytical column and a gradient of 0-15-45% B over 45 min. For preparative HPLC a similar gradient was used to afford the ligation product in ˜47% isolated yield (1.8 mg).

### Desulfurization:

The ubiquitinated peptide was desulfurized as described above to afford pure peptide 22 in 75% isolated yield (1.35 mg).

Analytical HPLC traces/(ESMS) of the ligation reaction between peptides (AVSEGTK)-Nbz and peptide 29 after 18 hours show peak c which corresponds to the ligated product with the observed mass of 10164.0 Da (calcd mass=10164.6 Da).

Analytical HPLC traces/(ESMS) of the ligation reaction between peptides (AVSEGTK)-Nbz and peptide 29 after desulfurization (after 10 hours) show peak d which corresponds to peptide 22 with the observed mass of 10100.9 Da (calcd mass=10098.7 Da) and Peak which corresponds to the thiol additives.

4biii) Synthesis of Ubiquitinated Peptide 23

Sequential Ligation: Peptide 29 (3.0 mg, 1 eq) was subjected to sequential ligation with peptide (GELAKHAVSEGTH)-Nbz (1.40, 3 eq). The peptides were dissolved in 105 μL of 6 M guanidine.HCl, 10 mM TCEP, 200 mM phosphate buffer pH ˜7.2. To this solution 2.1 μL each of benzylmercaptan and thiophenol were added and incubated for 18 h at 37° C. The reaction was followed using an analytical column and a gradient of 0-15-45% B over 45 min. For preparative HPLC a similar gradient was used to afford the ligation product in ˜45% yield (2.0 mg).

Desulfurization:

The ubiquitinated peptide was desulfurized as described above to afford 1 mg of pure peptide 23 (65% yield).

Analytical HPLC traces/(ESMS) of the ligation reaction between peptides (GELAKHAVSEGTK)-Nbz and peptide 29 after 18 hours show peak c and peak d, which correspond to the hydrolyzed and intramolecular cyclized peptide derived from peptide (GELAKHAVSEGTK)-Nbz, while peak e showed unknown mass. Peak f corresponds to the ligated product with the observed mass of 10800.4 Da (calcd mass=10798.4 Da).

Analytical HPLC traces/(ESMS) of the ligation reaction between peptides (GELAKHAVSEGTK)-Nbz and peptide 29 after desulfurization (after 10 hours) show peak d which corresponds to peptide 23 with the observed mass of 10735.9 Da (calcd mass=10734.3 Da) and Peak which corresponds to the thiol additives.

4biv) Synthesis of Ubiquitinated Peptide 24

Sequential Ligation: Peptide 29 (2.6 mg, 1 eq) was subjected to sequential ligation with peptide (IQTAVRLLLPGELARRAVSEGTK)-NEz (2.2, 3 eq). The peptides were dissolved in 91 μL of 6 M guanidine.HCl, 10 mM TCEP, 200 mM phosphate buffer pH ˜7.2. To this solution 1.8 μL each of benzylmercaptan and thiophenol were added and incubated for 18 h at 37° C. The reaction was followed using an analytical column and a gradient of 5-15-45% B over 45 min. For preparative HPLC a similar gradient was used to afford the ligation product in ˜44% yield (2.1 mg).

Desulfurization:

The ubiquitinated peptide was desulfurized as described above to afford 1.5 mg of pure peptide 24 (70% yield).

Analytical HPLC traces/(ESMS) of the ligation reaction between peptides (IQTAVRLLLPGELAKHAVSEGTK)-NBz and peptide 29 after 18 hours shows peak c, peak e, and peak f which corresponds to the hydrolyzed and intramolecularly cyclized peptide and benzylmercaptan exchanged thioester derived from peptide (IQTAVRLLLPGELAKRAVSEGTK)-NBz, while peak d showed unknown mass. Peak g corresponds to the ligated product with the observed mass of 11905.1 Da (calcd mass=11903.8 Da).

Analytical HPLC traces/(ESMS) of the ligation reaction between peptides (IQTAVRLLLPGELAKHAVSEGTK)-NBz and peptide 28 desulfurization (after 10 hours) shows peak h which corresponds to peptide 24 with the observed mass of 11841.6 Da (calcd mass=11839.7 Da) and Peak * which corresponds to the thiol additives.

4c) Evaluation of the UCH-L3 Activity with Peptides 22-24

The UCH-L3 activities with the ubiquitinated peptides 22-24 were assessed as described in the methods section above. As seen in FIG. 3, the peptides 22, and 23 with up to 21 amino acids gave a 65-70% hydrolysis within 30 minutes and were completely disassembled within 90 minutes. The ubiquitinated peptide 24, comprising of 31 residues, afforded 25% hydrolysis within 30 minutes and required 5 hours for a complete hydrolysis.

### Example 5

### Developing a High Throughput-Screening Assay for Deubiquitinases Based on the Expeditious Synthesis of Ubiquitinated Peptides

The synthesis of peptide 32 was carried out as described previously for peptides for peptides 17-20, however without applying the desulfurization step. The Dnp labeled aspartic acid that was used in the ubiquitin sequence we prepared according to J. Org. Chem., Vol. 72, No. 18, 2007, 6703, while the MCA that was introduced to the n-terminal of the was coupled by using 7-methoxycoumarinyl-4-acetic acid, DIC, HOBt (4 equiv each), DMF for 1 hour.

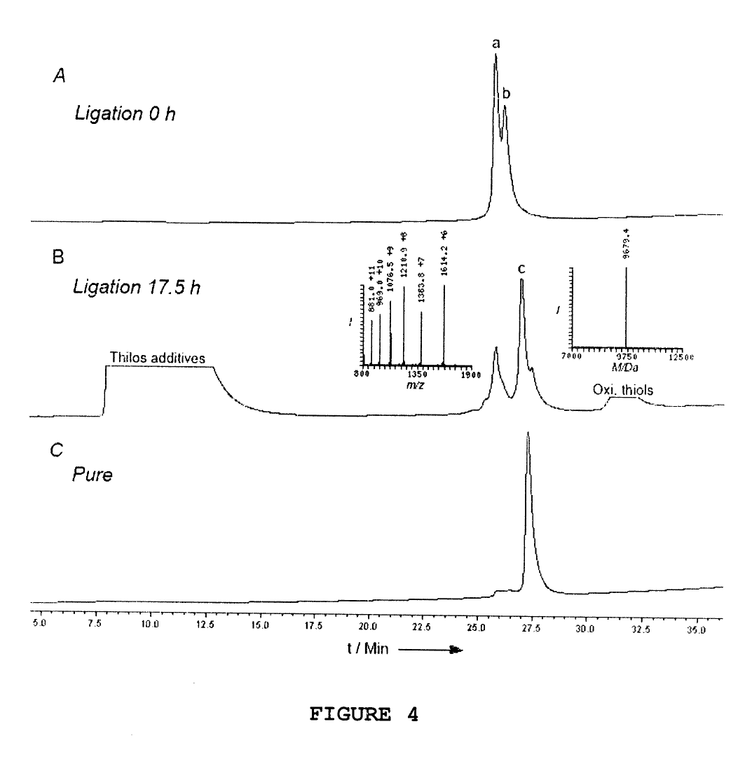

FIG. 4 shows the HPLC Data to prepare the reagent assay 32 with peak c representing the desired product 33 with the mass of 9679.4 Da. (b) while panel C shows the product after purification.

The obtained FRET system was next tested in a fluorescence-based assay. Initially, the fluorescence emission (Eₑₓ=325, Eₑₘ=445) was measured under folding conditions without any DUB. Under these conditions, the quenching efficiency of both ubiquitinated peptides was examined. The results show that complete quenching was observed. Following this, the changes in fluorescence were evaluated upon cleavage with the UCH-L3 by measuring the increase of fluorescence emission upon cleavage. Thus the substrate was treated with UCH-L3 for 30 minutes of in which nearly 6 fold increase in fluorescence was observed.

FIG. 5 is a FRET based assay for the UCH-L3 enzyme that we developed in this study. This figure shows as blue (circles, middle line) and magenta color (squares, bottom line) the effectiveness of the quenching properties of the FRET pair in the ubiquitinated peptide, while the green line (triangles, top line) shows the increase of fluorescence upon addition of the UCH-L3 enzyme.

1. A process for preparing ubiquinated peptide conjugates comprising a ubiquitin peptide residue UR attached at its C-terminus to a substrate peptide via a native isopeptide bond, said process comprising combining Native Chemical Ligation (NCL) and solid phase peptide synthesis (SPPS), by:
i) conducting Solid Phase Protein Synthesis (SPPS) to obtain peptide fragment A linked to a solid support: 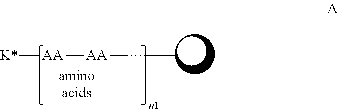
wherein said peptide fragment A contains n₁ amino acids (AA), n₁ being an integer ≧0, and said peptide fragment A N-terminating with a modified Lys amino acid K*: 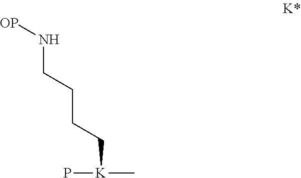
whereas:
P is a terminal-amine protecting group, and
OP is an orthogonal ε-amine protecting group,
further wherein K is a Lys amino acid backbone of the formula —(NHCH—C═O)—, said K amino acid forming a branching point for further elongation of said peptide fragment A;
ii) selectively removing said ε-amine orthogonal protecting group OP, and adding by SPPS up to m₁ amino acids belonging to a Ubiquitin peptide at the ε-amine of said Lys amino acid in peptide fragment A, optionally further adding by SPPS up to n₂ amino acids to said terminal amine of said Lys amino acid in peptide fragment A, wherein n₂ is an integer ≧0, m₁ is an integer ≧1 and n₁+n₂+m₁+1 is an integer ≦80,
further whereas said adding of up to n₂ amino acids to said terminal amine is conducted either before removing said OP group, or after the adding of up to m₁ amino acids belonging to a Ubiquitin peptide at the ε-amine,
thereby obtaining a ubiquinated-fragment peptide conjugate B linked to said solid support: 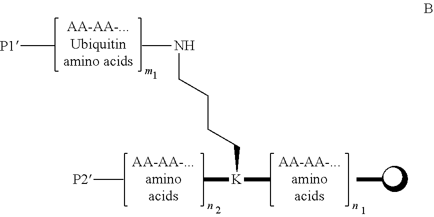
wherein said ubiquinated-fragment peptide conjugate B contains up to m₁ amino acids belonging to a Ubiquitin peptide at the e-amine of said Lys amino acid, up to n₁ amino acids attached to said solid support at the C-terminus of said Lys amino acid, prior to said branching point, and up to n₂ amino acids attached at the N-terminus of said Lys amino acid, after said branching point, further wherein said ubiquinated-fragment peptide conjugate B contains n₁+n₂+m₁+1 amino acids, and N-terminates with P1′ and P2′ amine-protecting groups;
iii) cleaving said ubiquinated-fragment peptide conjugate B from said solid support, to obtain a free ubiquinated-fragment peptide conjugate C: 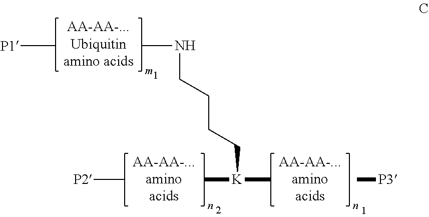
wherein said ubiquinated fragment peptide conjugate C contains n₁+n₂+m₁+1 amino acids, and terminates with P1′ and P2′ amine-protecting groups and a P3′ carboxy-protecting group;
iv) optionally further elongating said ubiquinated fragment peptide conjugate C by ligating it with one or more additional peptides, by Native Chemical Ligation (NCL), to obtain a ubiquinated peptide conjugate D: 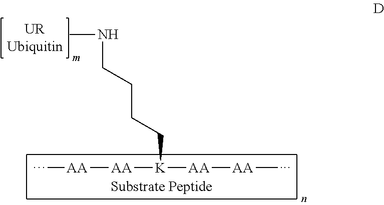
wherein said ubiquinated peptide conjugate D contains m amino acids of a ubiquitin peptide attached via an isopeptide bond to a substrate peptide containing n amino acids, such that m≧m₁ and n≧n₁+n₂+1.

2. The process of claim 1, wherein said OP group is selected from azide, allyloxycarbonyl (Alloc), 1-[4,4-dimethyl-2,6-dioxo-cyclohexylidene]-3-methylbutyl (IvDde), 1,1-dichloro-2,2-bis(p-chlorophenyl)ethylene (Dde) and 9-Fluorenylmethoxycarbonyl (FMOC).

3. The process of claim 2, whereas said SPPS is an Fmoc-SPPS, and said OP group is selected from azide, allyloxycarbonyl (Alloc), 1-[4,4-dimethyl-2,6-dioxo-cyclohexylidene]-3-methylbutyl (IvDde) and 1,1-dichloro-2,2-bis(p-chlorophenyl)ethylene (Dde).

4. The process of claim 2, whereas said SPPS is a Boc-SPPS, and said OP group is selected from azide, allyloxycarbonyl (Alloc), 1-[4,4-dimethyl-2,6-dioxo-cyclohexylidene]-3-methylbutyl (IvDde) and 9-Fluorenylmethoxycarbonyl (FMOC).

5. The process of claim 1, wherein said additional peptides being added during said native chemical ligation in step iv, are thioester peptides or thioester equivalent peptides.

6. The process of claim 1, wherein P, P1′ and P2′ are independently selected from hydrogen, benzyloxycarbonyl (Cbz), tert-butyloxycarbonyl (Boc), 9-fluorenylmethyloxycarbonyl (Fmoc), thiazolidine (THz), photolabile 2-nitro benzene and methylsulfonylethoxycarbonyl (MSC).

7. The process of claim 1, wherein P3′ is selected from hydrogen, N-acylurea (Nbz) and a latent thioester Functionality (LTF) residue having the general structure of Formula V: 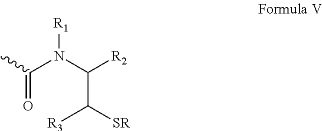
wherein:
R is either hydrogen or a thiol protecting group;
R₁ is selected from the group consisting of: hydrogen, C1-C3 alkyl, C1-C3 alkyl-COOH, C1-C3 alkyl-CONH₂, C1-C3 alkylene-CONH₂, C1-C3 alkylene-CO₂H, SO₂-alkyl; SO₂-alkyl-CONH₂, benzyl and derivatives thereof, alkyl-nitrile and alkyl-halogens;
R₂ and R₃ are selected from the group consisting of: hydrogen, CO₂H, CH₂CO₂H, —CH₂OH, CONH₂, CH₂—CONH₂ and CH₂NH₂, and N-protected derivatives thereof.

8. The process of claim 1, wherein in step ii thereof, the m₁ amino acid being added is a Cys amino acid, thereby forming a modified ubiquitin peptide fragment containing m₁ amino acids, and having a C-terminal Cys amino acid residue.

9. The process of claim 8, wherein said Cys amino acid residue has the general formula II: 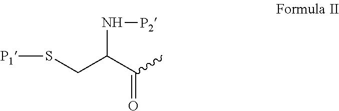
wherein at least one of said P₁′ or P₂′ on said Cys amino acid residue is selected from: thiazolidine (THz), photolabile 2-nitro benzene, and methylsulfonylethoxycarbonyl (MSC).

10. The process of claim 1, wherein said ubiquitin residue UR contains at least one labeled amino acid.

11. The process of claim 1, wherein said substrate peptide contains at least one labeled amino acid.

12. The process of claim 1, wherein said substrate peptide has a specific binding affinity with a species selected from an enzyme, an antigen, an agonist, an antibody, a lectin, and a carbohydrate.

13. The process of claim 12, wherein said enzyme is a deubiquetenase (Dub).

14. The process of claim 1, further comprising desulfurizing said ubiquinated peptide conjugate to convert any unnatural Cys amino-acids into the respective native Ala amino acids.

15. A Ubiquinated peptide conjugate comprising a ubiquitin peptide residue UR, attached at its C-terminus to a substrate peptide via a native isopeptide bond, said Ubiquinated peptide conjugate having the general structure of Formula I: 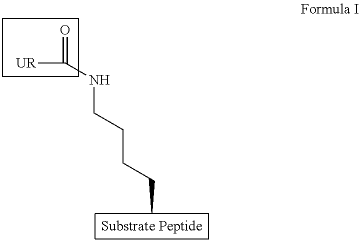
wherein said UR is selected from:
a) a fragment of a ubiquitin peptide;
b) a full-length modified ubiquitin peptide;
c) a fragment of a modified ubiquitin peptide.

16. The Ubiquinated peptide conjugate of claim 15, wherein said UR is a fragment of a modified ubiquitin peptide, having a Cys N-terminal amino acid residue, said Cys amino acid residue having the general formula II: 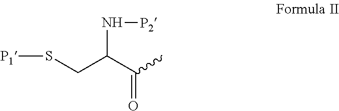
wherein at least one of said P₁′ or P₂′ on said Cys amino acid residue is selected from: thiazolidine (THz), photolabile 2-nitro benzene, and methylsulfonylethoxycarbonyl (MSC).

17. The ubiquinated peptide conjugate of claim 15, wherein said UR is a full-length modified ubiquitin peptide or a fragment of a modified ubiquitin peptide, containing at least one labeled amino acid.

18. The Ubiquinated peptide conjugate of claim 17, further wherein said substrate peptide attached to said UR contains at least one labeled amino acid.

19. The Ubiquinated peptide conjugates of claim 17, wherein said labeled amino acid contains at least one fluorescent agent.

20. The Ubiquinated peptide conjugate of claim 15, for use in conducting High Throughput Screening Assay for detecting Deubiquitenase (DUBs) inhibitors.

21. The Ubiquinated peptide conjugate of claim 15, for use in the preparation of antigens for developing linkage-specific antibodies.

22. The Ubiquinated peptide conjugate of claim 15, for use in the preparation of substrates for enzymatic elongation of ubiquitin.

23. The ubiquinated peptide conjugate Ubiquitin-H2B having a native isopeptide bond between said ubiquitin peptide and said H2B peptide.

24. A kit for conducting High Throughput Screening (HTS) assays for identifying potential inhibitors against one or more deubiquitinases (DUBs), said kit comprising:
a) a DUB;
b) a labeled ubiquinated peptide conjugate comprising a ubiquitin peptide residue UR, attached at its C-terminus to a substrate peptide via a native isopeptide bond, said ubiquinated polypeptide conjugate having the general structure of Formula I: 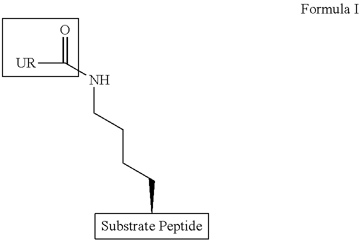
wherein said UR is a full-length ubiquitin peptide, or a fragment thereof, containing at least one labeled amino acid; and
c) an organic molecule to be tested as a possible inhibitor to said DUB.

25. The Ubiquinated peptide conjugates of claim 18, wherein said labeled amino acid contains at least one fluorescent agent.
